(19) United States
(12) Patent Application Publication
(10) Pub. No.: US 2002/0142950 A1
(43) Pub. Date: Oct. 3, 2002

(54) Methods for enhancing the bioavailability of a drug

(76) Inventors: Neil J. Hayward, North Grafton, MA (US); Malcolm L. Gefter, Lincoln, MA (US)

Correspondence Address: LAHIVE & COCKFIELD, 28 STATE STREET, BOSTON, MA, 02109, US

(21) Appl. No.: 09/781,133

(22) Filed: Feb. 9, 2001

Related U.S. Application Data
(60) Provisional application No. 60/181,833, filed on Feb. 11, 2000.
(60) Provisional application No. 60/181,943, filed on Feb. 11, 2000.

(51) Int. Cl. A61K 38/17; A61K 31/5415

(52) U.S. Cl. 514/12; 514/224.8

(57) ABSTRACT

The invention provides methods and compositions for enhancing the bioavailability of a drug in a subject. The present invention also provides methods and compositions for treating or preventing hepatic injury in a subject in need thereof. The invention further provides methods for identifying hydrophobic peptides, e.g., β-amyloid peptide derivatives, which are useful in enhancing bioavailability of a drug in a subject.

### RELATED APPLICATIONS

This application claims priority to U.S. provisional Application No. 60/181,833, filed on Feb. 11, 2000 and to U.S. provisional Application No. 60/181,943, filed on Feb. 11, 2000, the contents of each of which are incorporated herein in their entirety by this reference.

### FIELD OF THE INVENTION

This invention relates to methods for enhancing the bioavailability of a drug, e.g., the bioavailability of a drug to the brain or the oral bioavailability of a drug; methods for treating hepatic injury in a subject; as well as compounds useful in these methods.

### BACKGROUND OF THE INVENTION

Treatment of many diseases can be severely limited by resistance to the chosen therapeutic drug. For example, chemotherapy, while generally an effective treatment against human cancerous diseases, is hampered when a patient becomes resistant to the chemotherapeutic agent. In one form of drug resistance, called “multidrug resistance”, the cell becomes resistant not only to the specific chemotherapeutic agent being administered to the patient, but also to a wide range of structurally and functionally unrelated agents (see Ford et al., Pharmacological Reviews, 42:155-199, 1992).

The cause of multidrug resistance is the appearance of an integral glycoprotein in the plasma membrane of the affected cell. This glycoprotein functions as a multidrug transporter, and is variously called MultiDrug-Resistance 1 protein (MDR1), P-glycoprotein (pleiotropic-glycoprotein), Pgp, or P-170. P-glycoprotein consists of 1280 amino acid residues, and contains 12 transmembrane segments and two nucleotide-binding domains. P-glycoprotein strongly resembles prokaryotic and eukaryotic members of the so-called ATP (ATP Binding Cassette) transporters, or traffic ATPases (see Endicott et al., Annu. Rev. Biochem. 58:137-171, 1989; Higgins, Annu. Rev. Cell. biol 8:67-113, 1992).

P-glycoprotein is highly expressed in various normal tissues (e.g., the brain, intestine, lung, kidney, testis, and liver), and functions as an efflux pump for the cell. Consistent with its natural function, P-glycoprotein catalyses an ATP-dependent extrusion of various cytotoxic drugs from the cell, e.g., vinca alkaloids, anthracyclines, and other natural antibiotics, thereby maintaining their cellular level at a subtoxic concentration.

The phenomenon of multidrug resistance is not limited to tumor cells. P-glycoprotein and its homologues are expressed in a wide variety of cell-types, including parasitic protozoa. Consequently, overexpression of a member of the P-glycoprotein family of proteins creates obstacles to the treatment of a wide variety of parasitic diseases, including malaria, African sleeping sickness, and others (Campbell et al., Chemotherapy of Parasitic Diseases, Plenum Press:NY, 1986; Henderson et al., Mol. Cell. Biol. 12:2855-65, 1992).

P-glycoprotein is also expressed by endothelial cells of human capillary blood vessels at the blood-brain barrier and blood-testis barrier (Ford et al., supra, at 159). The blood-brain barrier is believed to restrict the entry of many compounds, including drugs whose site of action is within the brain, from entering the brain.

It is known that verapamil, a drug that blocks voltage-dependent calcium channels, stimulates the activity of P-glycoprotein-bound ATPase at a concentration of 1 to 20 μM (Sarkadi et al., J. Biol. Chem. 267:4854-4858, 1992). At this concentration verapamil blocks the extrusion of antitumor drugs, however, its high toxicity severely limits its clinical use (Solary et al., Leukemia 5:592-597, 1991; Dalton et al., J. Clin. Oncology 7:415-418, 1989). There is a need for additional compounds that are capable of enhancing the bioavailability of a drug in a subject.

### SUMMARY OF THE INVENTION

The present invention provides methods and compositions for enhancing the bioavailability of a drug in a subject based on administering a hydrophobic peptide to the subject in which the drug is also administered or is already present. The present invention also provides methods and compositions for treating or preventing hepatic injury in a subject in need thereof.

The present invention is based, at least in part, on the discovery that administration of a P-glycoprotein inhibitor to an animal, e.g., a rat, results in a decreased production of hepatic enzymes in the liver of the animal. The present invention is further based, at least in part, on the discovery that administration of a P-glycoprotein inhibitor to an animal, e.g., a rat, results in a decreased concentration of an administered drug, e.g., a hydrophobic peptide such as PPI-1019, in the liver of the animal (see, in particular, FIG. 6).

Accordingly, the invention features a method for enhancing the bioavailability or concentration of a drug in a subject, by administering to the subject a hydrophobic peptide, e.g., a β-amyloid peptide derivative, in an amount sufficient to enhance the bioavailability or concentration of the drug in the subject. In a preferred embodiment, the β-amyloid peptide derivative PPI-558, PPI-657, PPI-1019, PPI-578, or PPI-655 is administered to a subject to enhance the bioavailability or concentration of a drug in the subject (e.g., the bioavailability or concentration of a drug in the brain of the subject).

In yet another embodiment, the method of the invention includes administering to a subject a hydrophobic peptide, e.g., a β-amyloid peptide derivative, in combination with a P-glycoprotein inhibitor such as an antiarrhythmic agent, e.g., amiodarone or lidocaine; an antibiotic, e.g., cyclosporin or valspodar; an antifungal agent, e.g., cefoperazone; a calcium channel blocker, e.g., verapamil or felodipine; a chemotherapeutic agent, e.g., Taxol or Actinomycin D; a hormone, e.g., cortisol or tamoxifen; an antiparasite agent; a local anesthetic, e.g., aspirin; a phenothiazine; or a tricyclic antidepressant, e.g., Trazodone.

In yet another embodiment, the method of the invention includes administering to a subject a hydrophobic peptide, e.g., a β-amyloid peptide derivative in combination with a cytochrome P450 inhibitor such as calcium channel blockers, e.g., Verapamil, Felodipine, or Diltiazem; flavanoids, e.g., Quercetin, Kaempherol, or Benzoflavone; steroid hormones, e.g., cortisol, or progesterone; chemotherapeutic agents; or antidiabetic agents, e.g., Tolbutamide.

In preferred embodiments, the subject is a mammal, more preferably a human. In yet other preferred embodiments, the subject is suffering from a disorder, for example, a CNS disorder such as a neurodegenerative disorder, e.g., Alzheimer's disease, Parkinson's disease, multiple sclerosis, amyotrophic lateral sclerosis, progressive supranuclear palsy, epilepsy, Jakob-Creutzfieldt disease, or AIDS related dementia; cancer, e.g., glioblastoma; stroke; traumatic brain injury; or psychiatric disorders.

In a preferred embodiment, the drug whose bioavailability is enhanced, inhibits aggregation of natural β-amyloid peptide. In other preferred embodiments, the drug is an anti-cancer drug, e.g., a chemotherapeutic agent; an anti-inflammatory agent, e.g., nitric oxide, mannitol, allopurinol, or dimethyl sulfoxide; an anti-depressant; or a cholinestarase inhibitor.

In one embodiment of the method of the invention, the drug and the hydrophobic peptide, e.g., the β-amyloid peptide derivative, are administered to the subject orally, intravenously, intramuscularly, or subcutaneously, preferably in a pharmaceutically acceptable formulation. The pharmaceutically acceptable formulation is preferably a lipid-based formulation, a saline based formulation, or a manitol based formulation. The drug and the hydrophobic peptide, e.g., the β-amyloid peptide derivative, can be administered in the same formulation or in separate formulations. In other preferred embodiments, the drug and the hydrophobic peptide, e.g., the β-amyloid peptide derivative, are administered simultaneously. In yet other preferred embodiments, the drug and the hydrophobic peptide, e.g., the β-amyloid peptide derivative, are administered at different times. For example, the drug can be administered every 2, 4, 6, 8, 10, 12, or 24 hours, and the hydrophobic peptide, e.g., the β-amyloid peptide derivative, can be administered every 2, 4, 6, 8, 10, 12, or 24 hours, wherein the time of administration of the drug and the hydrophobic peptide may be the same or different.

In another aspect, the invention features a method for enhancing the oral bioavailability of a drug. The method includes administering to a subject a hydrophobic peptide, e.g., a β-amyloid peptide derivative, in an amount sufficient to enhance the oral bioavailability of the drug, transportation of the drug across the gastrointestinal tract, and entry into the bloodstream, thereby enhancing the oral bioavailability of the drug.

In yet another aspect, the invention features a method for treating Alzheimer's disease in a subject. The method includes administering to the subject a hydrophobic peptide, e.g., a β-amyloid peptide derivative, and, optionally, a drug, e.g., a drug which inhibits aggregation of natural β-amyloid peptide, in amounts sufficient to treat Alzheimer's disease in the subject.

In a further aspect, the invention features a method for enhancing the bioavailability of a β-amyloid peptide derivative to the brain of a subject, e.g., the uptake of the peptide into the brain of the subject. The method includes administering to the subject the β-amyloid peptide derivative and a P-glycoprotein inhibitor, thereby enhancing the bioavailability of the β-amyloid peptide derivative to the brain of the subject. In one embodiment, the β-amyloid peptide derivative is PPI-558, PPI-657, PPI-1019, PPI-578, or PPI-655. In another embodiment, the P-glycoprotein inhibitor is cyclosporin or valspodar. In another embodiment, the method further includes administering to the subject a cytochrome P450 inhibitor, in addition to or instead of the P-glycoprotein inhibitor.

The β-amyloid peptide derivative and the P-glycoprotein inhibitor can be administered in the same formulation or in separate formulations. In one embodiment, the β-amyloid peptide derivative and the P-glycoprotein inhibitor are administered simultaneously. In another embodiment, the β-amyloid peptide derivative and the P-glycoprotein inhibitor are administered at different times. For example, the β-amyloid peptide derivative can be administered every 2, 4, 6, 8, 10, 12, or 24 hours, and the P-glycoprotein inhibitor can be administered every 2, 4, 6, 8, 10, 12, or 24 hours, wherein the time of administration of the peptide and the inhibitor may be the same or different.

In another aspect, the invention features a pharmaceutical composition for enhancing the bioavailability of a drug which includes a hydrophobic peptide, e.g, a β-amyloid peptide derivative, and a drug. Another embodiment features a pharmaceutical composition including a hydrophobic peptide, e.g., a β-amyloid peptide derivative, and a P-glycoprotein inhibitor. Yet another embodiment features a pharmaceutical composition including a hydrophobic peptide, e.g., a β-amyloid peptide derivative, and a cytochrome P450 inhibitor. Such compositions can further include a pharmaceutically acceptable carrier.

In yet another aspect, the invention features a method for identifying a hydrophobic peptide, e.g., a β-amyloid peptide derivative, capable of increasing the bioavailability, e.g., the bioavailability in the brain or oral bioavailability, of a drug in a subject. The method includes screening a candidate hydrophobic peptide for the ability to bind to P-glycoprotein and inhibit its function, and selecting a hydrophobic peptide which binds to P-glycoprotein and inhibits its function, thereby identifying a hydrophobic peptide capable of increasing bioavailability of a drug in a subject.

In yet a further aspect, the invention features a method for identifying a hydrophobic peptide, e.g., a β-amyloid peptide derivative, capable of increasing the bioavailability, e.g., the bioavailability in the brain or oral bioavailability, of a drug in a subject which includes screening a candidate hydrophobic peptide for the ability to bind to cytochrome P450 and inhibit its function, and selecting a hydrophobic peptide which binds to cytochrome P450 and inhibits its function, thereby identifying a hydrophobic peptide capable of increasing bioavailability of a drug in a subject.

In another aspect, the invention features, a kit which includes a hydrophobic peptide, e.g., a β-amyloid peptide derivative, and instructions for use in increasing the bioavailability of a drug in a subject. In another embodiment, the kit can further include a drug and/or a P-glycoprotein inhibitor and/or a cytochrome P450 inhibitor.

The present invention also provides a method for treating or preventing hepatic injury in a subject in need thereof. The method includes administering to the subject a P-glycoprotein inhibitor in an amount effective to treat or prevent hepatic injury in the subject. The method can also involve selecting a subject in need of treatment for or prevention of hepatic injury, prior to the administration of the P-glycoprotein inhibitor to the subject.

In one embodiment, the method of the invention includes administering to the subject a P-glycoprotein inhibitor such as an antiarrhythmic agent, e.g., amiodarone or lidocaine; an antibiotic, e.g., cyclosporin or valspodar; an antifungal agent, e.g., cefoperazone; a calcium channel blocker, e.g., verapamil or felodipine; a chemotherapeutic agent, e.g., Taxol or Actinomycin D; a hormone, e.g., cortisol or tamoxifen; an antiparasite agent; a local anesthetic, e.g., aspirin; a phenothiazine; or a tricyclic antidepressant, e.g., Trazodone. In another embodiment, the method of the invention includes administering to a subject a P-glycoprotein inhibitor in combination with a cytochrome P450 inhibitor such as a calcium channel blocker, e.g., Verapamil, Felodipine, or Diltiazem; a flavanoid, e.g., Quercetin, Kaempherol, or Benzoflavone; steroid hormones, e.g., cortisol, or progesterone; chemotherapeutic agents; or an antidiabetic agent, e.g., Tolbutamide.

In another embodiment, the hepatic injury is hepatic fibrosis, hepatic cirrhosis, hepatic injury caused by a drug, hepatic injury due to prolonged ethanol uptake, or hepatic injury due to carbon tetrachloride exposure.

In yet another embodiment, the P-glycoprotein inhibitor and the cytochrome P450 inhibitor are administered to the subject orally, intravenously, intramuscularly, or subcutaneously, preferably in a pharmaceutically acceptable formulation. The pharmaceutically acceptable formulation is preferably a lipid-based formulation, a saline based formulation, or a manitol based formulation. The P-glycoprotein inhibitor and the cytochrome P450 inhibitor can be administered in the same formulation or in separate formulations. In other preferred embodiments, the P-glycoprotein inhibitor and the cytochrome P450 inhibitor are administered simultaneously. In yet other preferred embodiments, the P-glycoprotein inhibitor and the cytochrome P450 inhibitor are administered at different times.

In a further embodiment, the hepatic injury is caused by a drug and the P-glycoprotein inhibitor is administered to the subject simultaneously with the drug, within 1, 2, 3, 4, 5, 6, 7, 8, 9, 10, 11, or 12 hours after the drug is administered to the subject, or within 1, 2, 3, 4, 5, 6, 7, 8, 9, 10, 11, or 12 hours before the drug is administered to the subject. In one embodiment, the drug is a hydrophobic peptide such as a β-amyloid peptide derivative, e.g., PPI-558, PPI-657, PPI-1019, PPI-578, or PPI-655.

In yet another embodiment, the hepatic injury is due to carbon tetrachloride exposure, and the P-glycoprotein inhibitor is administered to the subject within 1, 2, 3, 4, 5, 6, 7, 8, 9, 10, 11, 12, 13, 14, 15, 16, 17, 18, 19, 20, 21, 22, 23, or 24 hours after the carbon tetrachloride exposure.

In another embodiment, the P-glycoprotein inhibitor is administered to the subject in an amount of 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, or 65 mg/kg. In a preferred embodiment, the P-glycoprotein inhibitor valspodar is administered to the subject in an amount of 12.5 mg/kg. In another preferred embodiment, the P-glycoprotein inhibitor cyclosporin is administered to the subject in an amount of 50 mg/kg.

In preferred embodiments, the subject is a mammal, such as a rat, a mouse, or more preferably a human.

In another aspect, the invention features a method for modulating, e.g., decreasing, the levels of a hepatic enzyme in a subject. The method includes administering to the subject a P-glycoprotein inhibitor in an amount effective to modulate the levels of a hepatic enzyme in the subject. The method can also involve selecting a subject in need of modulation of hepatic enzymes, prior to the administration of the P-glycoprotein inhibitor to the subject.

In one embodiment, the hepatic enzyme is alanine aminotransferase, aspartate aminotransferase, or γ-glutammyl transferase.

In another aspect, the invention features a pharmaceutical composition including a P-glycoprotein inhibitor and a drug, wherein the drug is present in an amount effective to treat a targeted condition in a subject and the P-glycoprotein inhibitor is present in an amount effective to prevent hepatic injury in the subject. In one embodiment, the pharmaceutical composition further includes a cytochrome P450 inhibitor. In another embodiment, the pharmaceutical composition further includes a pharmaceutically acceptable carrier, e.g., a lipid-based carrier.

In another aspect, the invention features a kit including a P-glycoprotein inhibitor, a drug, and instructions for administration to a subject in an amount effective to treat a targeted condition in the subject and prevent hepatic injury in the subject. In one embodiment, the kit further includes a cytochrome P450 inhibitor.

Other features and advantages of the invention will be apparent from the following detailed description, and from the claims.

### BRIEF DESCRIPTION OF THE DRAWINGS

FIG. 1 is a graph depicting the levels of PPI-558 in the brain parenchyma and the brain capillaries, following intra-arterial administration in the rat.

FIG. 2 is a graph depicting the effects of cyclosporin A on the levels of PPI-558 in the brain of a rat.

FIG. 3 is a graph depicting the effects of cyclosporin A on the plasma levels of PPI-558 in the rat.

FIG. 4 is a graph depicting the effects of cyclosporin A on the levels of PPI-1019 in the brain of a rat.

FIG. 5 is a graph depicting the effects of cyclosporin A on the plasma levels of PPI-1019 in the rat.

FIG. 6 is a graph depicting the biodistribution of tritiated PPI-1019 in the presence or the absence of cyclosporin A.

### DETAILED DESCRIPTION

The present invention provides methods and compositions for enhancing the bioavailability of a drug in a subject, as well methods and compositions for treating or preventing hepatic injury in a subject in need thereof.

Accordingly, in one aspect, the invention features a method for enhancing or increasing the bioavailability of a drug in a subject, in which a hydrophobic peptide, e.g., a β-amyloid peptide derivative, is administered in an amount sufficient to enhance bioavailability of the drug in the subject (e.g., enhance delivery of the drug across the blood brain barrier and entry into the brain).

In another aspect, the present invention features a method for treating or preventing hepatic injury in a subject in need thereof. The method includes administering to the subject a P-glycoprotein inhibitor in an amount effective to treat or prevent hepatic injury in the subject. The method can also involve selecting a subject in need of treatment for or prevention of hepatic injury, prior to the administration of the P-glycoprotein inhibitor to the subject.

As used herein, the term “hepatic injury” includes an injury to the liver, such as an injury to the liver that interferes with the normal function of the liver. The term hepatic injury includes an injury due to the over- or under-production of hepatic enzymes, e.g., alanine aminotransferase, aspartate aminotransferase, or γ-glutammyl transferase, in the liver. For example, the hepatic injury is hepatic fibrosis, hepatic cirrhosis, hepatic injury caused by a drug, hepatic injury due to prolonged ethanol uptake, or hepatic injury due to carbon tetrachloride exposure.

As used herein, the term “hydrophobic peptide” includes a hydrophobic peptide which has the ability to enhance or increase the bioavailability of a drug in a subject. The term hydrophobic peptide includes peptides, e.g., hydrophobic peptides comprised of L-amino acids, as well as peptide analogs, peptide derivatives, and peptide mimetics, e.g., hydrophobic peptides comprised of D-amino acids with the proviso that the term “peptide” is not intended to include the compounds described in U.S. Pat. Nos. 5,543,423 and 5,723,459 comprising amino acid derivatives. The terms “peptide analog”, “peptide derivative” and “peptide mimetic” as used herein are intended to include molecules which mimic the chemical structure of a peptide and retain the functional properties of the peptide. Approaches to designing peptide analogs are known in the art. For example, see Farmer, P. S. in Drug Design (E. J. Ariens, ed.) Academic Press, New York, 1980, vol. 10, pp. 119-143; Ball. J. B. and Alewood, P. F. (1990) J. Mol. Recognition 3:55; Morgan, B. A. and Gainor, J. A. (1989) Ann. Rep. Med. Chem. 24:243; and Freidinger, R. M. (1989) Trends Pharmacol. Sci. 10:270.

The hydrophobic peptide analogs or mimetics of the invention also include isosteres. The term “isostere” as used herein refers to a sequence of two or more residues that can be substituted for a second sequence because the steric conformation of the first sequence fits a binding site specific for the second sequence. The term specifically includes peptide back-bone modifications (i.e., amide bond mimetics) well known to those skilled in the art. Such modifications include modifications of the amide nitrogen, the α-carbon, amide carbonyl, complete replacement of the amide bond, extensions, deletions or backbone crosslinks. Several peptide backbone modifications are known, including Ψ[CH₂S], Ψ[CH₂NH], Ψ[C(S)NH₂], Ψ[NHCO], Ψ[C(O)CH₂], and Ψ[(E) or (Z) CH═CH]. In the nomenclature used above, Ψ indicates the absence of an amide bond. The structure that replaces the amide group is specified within the brackets. Other examples of isosteres include peptides substituted with one or more benzodiazepine molecules (see e.g., James, G. L. et al. (1993) Science 260:1937-1942).

Other possible modifications include an N-alkyl (or aryl) substitution (Ψ[CONR]), backbone crosslinking to construct lactams and other cyclic structures, or retro-inverso amino acid incorporation (Ψ[NHCO]). By “inverso” is meant replacing L-amino acids of a sequence with D-amino acids, and by “retro-inverso” or “enantio-retro” is meant reversing the sequence of the amino acids (“retro”) and replacing the L-amino acids with D-amino acids. For example, if the parent peptide is Thr-Ala-Tyr, the retro modified form is Tyr-Ala-Thr, the inverso form is thr-ala-tyr, and the retro-inverso form is tyr-ala-thr (lower case letters refer to D-amino acids). Compared to the parent peptide, a retro-inverso peptide has a reversed backbone while retaining substantially the original spatial conformation of the side chains, resulting in a retro-inverso isomer with a topology that closely resembles the parent peptide and is able to bind the selected LHRH receptor. See Goodman et al. “Perspectives in Peptide Chemistry” pp. 283-294 (1981). See also U.S. Pat. No. 4,522,752 by Sisto for further description of “retro-inverso” peptides.

In a preferred embodiment, the hydrophobic peptide includes about 30-40 amino acids, preferably about 20-30 amino acids, more preferably about 10-20 amino acids, and most preferably about 4, 5, 6, 7, 8, 9, or 10 amino acids. Preferably, the hydrophobic peptide includes at least 50%, preferably 60%, more preferably 70%, even more preferably 80%, and most preferably 90%, 95% or more hydrophobic amino acids, e.g., leucines, valines, isoleucines, tyrosines, or tryptophans. In a preferred embodiment, the peptide is a β-amyloid peptide derivative.

As used herein, the term “β-amyloid peptide derivative” includes peptides derived from the natural β-amyloid peptide (β-AP). Natural β-AP is derived by proteolysis of a larger protein called the amyloid precursor protein (APP) described in Kang, J. et al. (1987) Nature 325:733; Goldgaber, D. et al. (1987) Science 235:877; Robakis, N. K. et al. (1987) Proc. Natl. Acad. Sci. USA 84:4190; Tanzi, R. E. et al. (1987) Science 235:880. Differential splicing of the APP messenger RNA leads to at least five forms of APP, composed of either 563 amino acids (APP-563), 695 amino acids (APP-695), 714 amino acids (APP-714), 751 amino acids (APP-751) or 770 amino acids (APP-770). Within APP, naturally-occurring β amyloid peptide begins at an aspartic acid residue at amino acid position 672 of APP-770. Naturally-occurring β-AP derived from proteolysis of APP is 39 to 43 amino acid residues in length, depending on the carboxy-terminal end point, which exhibits heterogeneity. The predominant circulating form of β-AP in the blood and cerebrospinal fluid of both AD patients and normal adults is β1-40 (“short β”). Seubert, P. et al. (1992) Nature 359:325; Shoji, M. et al. (1992) Science 258:126. However, β1-42 and β1-43 (“long β”) also are forms in β-amyloid plaques. Masters, C. et al. (1985) Proc. Natl. Acad. Sci. USA 82:4245; Miller, D. et al. (1993) Arch. Biochem. Biophys. 301:41; Mori, H. et al. (1992) J. Biol. Chem. 267:17082. β-amyloid peptide derivatives are described in detail in subsection I below and also described in PCT Application Nos. WO 96/28471 and WO 98/08868, the contents of which are incorporated herein by reference.

Not wishing to be bound by theory, it is believed that the peptides, e.g., the β-amyloid peptide derivatives, act to enhance bioavailability of a drug by inhibiting either or both of P-glycoprotein and cytochrome P450.

As used herein, the term “P-glycoprotein” includes an integral glycoprotein which is found in the plasma membrane of a cell, e.g., an endothelial cell, and is capable of functioning as a multidrug transporter (also known as MultiDrug-Resistance 1 protein (MDR1), pleiotropic-glycoprotein, Pgp, or P-170). Preferably, the P-glycoprotein is approximately 1280 amino acid residues, and contains 12 transmembrane segments and two nucleotide-binding domains. The P-glycoprotein strongly resembles prokaryotic and eukaryotic members of the so-called ATP (ATP Binding Cassette) transporters, or traffic ATPases (Endicott et al., Annu. Rev. Biochem. 58:137-171, 1989; Higgins, Annu. Rev. Cell. Biol. 8:67-113, 1992). P-glycoprotein is described in, for example, Mayer U. et al. J. Clin. Invest. 100(10):2430-2436, the contents of which are incorporated herein by reference.

As used herein, the term “P-glycoprotein inhibitor” includes compounds which have the ability to inhibit P-glycoprotein function. Such P-glycoprotein inhibitors are known in the art and include antiarrhythmic agents, antibiotics, antifungal agents, calcium channel blockers, chemotherapeutic agents, hormones, antiparasitic agents, local anesthetics, phenothiazines, and tricyclic antidepressants. P-glycoprotein inhibitors are described in, for example, U.S. Pat. Nos. 5,567,592, 5,776,939, and PCT Application No. WO 95/31474, the contents of which are incorporated herein by reference. In one embodiment, the P- glycoprotein inhibitor is a hydrophobic peptide, such as a β-amyloid peptide derivative. Preferred P-glycoprotein inhibitors include cyclosporin A and valspodar.

As used herein, the term “cytochrome P450” includes members of the cytochrome P450 family, e.g., CPY1, CYP2, and CYP3, which are involved in drug metabolism. Cytochrome P450 family members can be found in the liver as well as in the enterocytes lining the lumen of the intestine. Several of the cytochrome P450 family members are inducible, i.e., their concentration as well as their catalytic activity is increased after exposure of an individual to particular classes of drugs, endogenous compounds, and environmental agents. Cytochrome P450 family members are described in, for example, Watkins P. B. et al. (1992) Gastroenterology Clinics of North America 21(3):511-526, the contents of which are incorporated herein by reference.

As used herein, the term “cytochrome P450 inhibitor” includes compounds which have the ability to inhibit cytochrome P450 function. Such cytochrome P450 inhibitors are known in the art and include calcium channel blockers, e.g., Verapamil, Felodipine, or Diltiazem; flavanoids, e.g., Quercetin, Kaempherol, or Benzoflavone; steroid hormones, e.g., cortisol, or progesterone; chemotherapeutic agents; or antidiabetic agents, e.g., Tolbutamide. Cytochrome P450 inhibitors are described in, for example, PCT Application No. WO 95/20980, the contents of which are incorporated herein by reference.

As used herein, the term “drug” is intended to encompass all types of pharmaceutical compounds and includes agents suitable for treating a targeted condition in a subject, e.g., a targeted condition of the brain, and capable of being delivered in active form, in vivo using the methods of the invention. The ordinarily skilled artisan would be able to select appropriate art-recognized drugs for a particular disease or condition targeted for treatment. Examples of such drugs include antibiotics, enzymes, chemical compounds, mixtures of chemical compounds, biological macromolecules, e.g., peptides, and analogs thereof. Similar substances are known or can be readily ascertained by one of skill in the art. One skilled in the art can look to Harrison's Principles of Internal Medicine, Thirteenth Edition, Eds. T. R. Harrison et al. McGraw-Hill N.Y., N.Y.; and the Physicians Desk Reference 50th Edition 1997, Oradell N.J., Medical Economics Co., the complete contents of which are expressly incorporated herein by reference, to determine appropriate drugs for administration to a subject.

In a preferred embodiment of the invention, the drug is a hydrophobic peptide such as a β-amyloid peptide derivative, e.g., PPI-558, PPI-657, PPI-1019, PPI-578, or PPI-655 (e.g., the drug is capable of inhibiting P-glycoprotein and/or cytochrome P450, as well as treating an underlying disorder in a subject, e.g., a CNS disorder). In one embodiment, the hydrophobic peptide, e.g., a β-amyloid peptide derivative, is administered via the internal carotid artery. In another embodiment, the hydrophobic peptide, e.g., a β-amyloid peptide derivative, is administered at a concentration sufficient to achieve brain levels of 100 nM, 150 nM, 200 nM, 250 nM, or more.

As used herein, the term “subject” includes animals which express P-glycoprotein and/or cytochrome P450 in, for example, their epithelial cells, e.g., the epithelial cells of the brain, liver, pancreas, small intestine, colon, kidney, testis, or adrenal gland, preferably mammals, most preferably humans. In a preferred embodiment, the subject is a primate, preferably a human. Other examples of subjects include mice, rats, dogs, cats, goats, and cows.

As used herein, the term “bioavailability” refers to the availability, amount (e.g., concentration), or pharmacological activity of a drug in a biological fluid, cell, or tissue, e.g., blood, serum, cerebrospinal fluid, or brain in a mammal, e.g., a human. As used herein, the term “enhancing the bioavailability” of a drug includes increasing or improving the availability, amount (e.g., concentration) or pharmacological activity of a drug in a biological fluid, cell, or tissue. This can be achieved by, for example, increasing the concentration of the drug in the targeted biological fluid, cell, or tissue, e.g., as compared to the concentration of the drug in the targeted biological fluid in the absence of the hydrophobic peptide. In one preferred embodiment, the concentration of the drug is higher in the targeted biological fluid, cell, or tissue that it would be in the absence of the hydrophobic peptide, e.g., the β-amyloid peptide derivative. The enhanced bioavailability of a drug in a subject can be determined by art known techniques. For example, a biological fluid, e.g., plasma or cerebrospinal fluid, can be removed from the subject (e.g., using a syringe) and the concentration of the drug in the biological fluid can be determined by, for example, using HPLC. The enhanced bioavailability of a drug in a subject can also be determined by detecting alleviation of the condition targeted for treatment by the drug. For example, the formation of amyloid deposits in the brain of the subject can be determined using the assays described herein.

As used herein, the term “blood brain barrier” is intended to include the endothelial lining of cells which are selectively permeable or impermeable to substances circulating outside of the brain.

In preferred embodiments, the subject is a mammal, more preferably a human. In other preferred embodiments, the subject is suffering from a disorder, e.g., a CNS disorder, a hepatic injury, a disorder characterized by multidrug resistance, a cardiovascular disorder, or a neuromuscular disorder. As used herein, the term “CNS disorder” includes a disease disorder or condition affecting the central nervous system, e.g., the brain. Examples of CNS disorders include neurodegenerative disorders, e.g., Alzheimer's disease, Parkinson's disease, multiple sclerosis, amyotrophic lateral sclerosis, progressive supranuclear palsy, epilepsy, Jakob-Creutzfieldt disease, or AIDS related dementia; cancer, e.g., glioblastoma; stroke; traumatic brain injury; or psychiatric disorders.

As used herein, the term “stroke” is art recognized and is intended to include sudden diminution or loss of consciousness, sensation, and voluntary motion caused by rapture or obstruction (e.g. by a blood clot) of an artery of the brain.

As used herein, the term “Traumatic Brain Injury” is art recognized and is intended to include the condition in which, a traumatic blow to the head causes damage to the brain, often without penetrating the skull. Usually, the initial trauma can result in expanding hematoma, subarachnoid hemorrhage, cerebral edema, raised intracranial pressure (ICP), and cerebral hypoxia, which can, in turn, lead to severe secondary events due to low cerebral blood flow (CBF).

As used herein, the term “hepatic enzyme” includes an enzyme that is secreted and/or functions in the liver. For example, the hepatic enzyme can be alanine aminotransferase, aspartate aminotransferase, or γ-glutammyl transferase.

Various aspects of the invention are described further in the following subsections.

I. P-glycoprotein Inhibitors

P-glycoprotein inhibitors intended to be used in the methods of the invention include compounds which have the ability to inhibit P-glycoprotein function. Such P-glycoprotein inhibitors are known in the art and include antiarrhythmic agents, antibiotics, antifungal agents, calcium channel blockers, chemotherapeutic agents, hormones, antiparasitic agents, local anesthetics, phenothiazines, and tricyclic antidepressants. P-glycoprotein inhibitors are described in, for example, U.S. Pat. Nos. 5,567,592, 5,776,939, and PCT Application No. WO 95/31474, the contents of which are incorporated herein by reference. In one embodiment, the P-glycoprotein inhibitor is a hydrophobic peptide, such as a β-amyloid peptide derivative. Other preferred P-glycoprotein inhibitors include cyclosporin A and valspodar.

II. β-amyloid Peptide Derivatives

Preferred hydrophobic peptides of the invention comprise a β-amyloid peptide derivative. β-amyloid peptide derivatives are described in, for example, PCT Application Nos. WO 96/28471 and WO 98/08868, the contents of which are incorporated herein by reference.

In one embodiment, the β-amyloid peptide derivative can be a β-amyloid peptide which binds to and inhibits the function of P-glycoprotein and/or cytochrome P450. Preferably, the β-amyloid peptide derivative is comprised of 3-20 D-amino acids or L-amino acids, more preferably 3-10 D-amino acids or L-amino acids, and even more preferably 3-5 D-amino acids or L-amino acids. In one embodiment, the β-amyloid peptide derivative is amino-terminally modified, for example with a modifying group comprising an alkyl group such as a C1-C6 lower alkyl group, e.g., a methyl, ethyl, or propyl group; or a cyclic, heterocyclic, polycyclic or branched alkyl group. Examples of suitable N-terminal modifying groups are described further in subsection II below. In another embodiment, the β-amyloid peptide derivative is carboxy-terminally modified, for example the β-amyloid peptide derivative can comprise a peptide amide, a peptide alkyl or aryl amide (e.g., a peptide phenethylamide) or a peptide alcohol. Examples of suitable C-terminal modifying groups are described further in subsections II and III below. The β-amyloid peptide derivative may be modified to enhance the ability of the β-amyloid peptide derivative to inhibit P-glycoprotein and/or alter β-AP aggregation or neurotoxicity. Additionally or alternatively, β-amyloid peptide derivatives may be modified to alter a pharmacokinetic property of the β-amyloid peptide derivative and/or to label the β-amyloid peptide derivative with a detectable substance (described further in subsection III below).

In another embodiment, a β-amyloid peptide derivative of the invention comprises a retro-inverso isomer of a β-amyloid peptide, wherein the β-amyloid peptide derivative binds to P-glycoprotein and inhibits its function and/or binds natural β-amyloid peptides or modulates the aggregation or inhibits the neurotoxicity of natural β-amyloid peptides when contacted with the natural β-amyloid peptides. Preferably, the retro-inverso isomer of the β-amyloid peptide derivative is comprised of 3-20 D-amino acids, more preferably 3-10 D-amino acids, and even more preferably 3-5 D-amino acids. In one embodiment, the retro-inverso isomer is amino-terminally modified, for example with a modifying group comprising an alkyl group such as a C1-C6 lower alkyl group, or a cyclic, heterocyclic, polycyclic or branched alkyl group. Examples of suitable N-terminal modifying groups are described further in subsection II below. In another embodiment, the retro-inverso isomer is carboxy-terminally modified, for example with an amide group, an alkyl or aryl amide group (e.g., phenethylamide) or a hydroxy group (i.e., the reduction product of a peptide acid, resulting in a peptide alcohol). Examples of suitable C-terminal modifying groups are described further in subsections II and III below. The retro-inverso isomer may be modified to enhance the ability of the β-amyloid peptide derivative to inhibit P-glycoprotein and/or cytochrome P450 function, and/or to alter β-AP aggregation or neurotoxicity. Additionally or alternatively, the retro-inverso isomer may be modified to alter a pharmacokinetic property of the β-amyloid peptide derivative and/or to label the β-amyloid peptide derivative with a detectable substance (described further in subsection III below).

The β-amyloid peptide derivatives of the invention preferably are designed based upon the amino acid sequence of a subregion of natural β-AP. The term “subregion of a natural β-amyloid peptide” is intended to include amino-terminal and/or carboxy-terminal deletions of natural β-AP. The term “subregion of natural β-AP” is not intended to include full-length natural β-AP (i.e., “subregion” does not include Aβ₁₋₃₉, Aβ₁₋₄₀, Aβ₁₋₄₁, Aβ₁₋₄₂ and Aβ₁₋₄₃). A preferred subregion of natural β-amyloid peptide is an “Aβ aggregation core domain” (ACD). As used herein, the term “Aβ aggregation core domain” refers to a subregion of a natural β-amyloid peptide that is sufficient to inhibit P-glycoprotein and/or cytochrome P450 function, and/or to modulate aggregation of natural β-APs when this subregion, in its L-amino acid form, is appropriately modified (e.g., modified at the amino-terminus), as described in detail in, for example, PCT Application No. WO98/08868, the entire content of which is expressly incorporated herein by reference. Preferably, the ACD is modeled after a subregion of natural β-AP that is less than 15 amino acids in length and more preferably is between 3-10 amino acids in length. In various embodiments, the ACD is modeled after a subregion of β-AP that is 10, 9, 8, 7, 6, 5, 4 or 3 amino acids in length. In one embodiment, the subregion of β-AP upon which the ACD is modeled is an internal or carboxy-terminal region of β-AP (i.e., downstream of the amino-terminus at amino acid position 1). In another embodiment, the ACD is modeled after a subregion of β-AP that is hydrophobic. Preferred Aβ aggregation core domains encompass amino acid residues 17-20 or 17-21 of natural β-AP (Aβ₁₇₋₂₀ and Aβ₁₇₋₂₁, respectively). The amino acid sequences of Aβ₁₇₋₂₀ and Aβ₁₇₋₂₁ are Leu-Val-Phe-Phe (SEQ ID NO:1) and Leu-Val Phe-Phe-Ala (SEQ ID NO:2), respectively.

D-amino acid-containing β-amyloid peptide derivatives designed based upon the amino acid sequences of Aβ₁₇₋₂₀ and Aβ₁₇₋₂₁ are particularly effective inhibitors of Aβ aggregation. These β-amyloid peptide derivatives can comprise a D-amino acid sequence corresponding to the L-amino acid sequence of Aβ₁₇₋₂₀ or Aβ₁₇₋₂₁, a D-amino acid sequence which is a retro-inverso isomer of the L-amino acid sequence of Aβ₁₇₋₂₀ or Aβ₁₇₋₂₁, or a D-amino acid sequence that is a scrambled or substituted version of the L-amino acid sequence of Aβ₁₇₋₂₀ or Aβ₁₇₋₂₁. The D-amino acid-based β-amyloid peptide derivatives may have unmodified amino- and/or carboxy-termini or, alternatively, the amino-terminus, the carboxy-terminus, or both, may be modified (described further below). The peptidic structures of effective β-amyloid peptide derivatives generally are hydrophobic and are characterized by the presence of at least two D-amino acid structures independently selected from the group consisting of a D-leucine structure, a D-phenylalanine structure and a D-valine structure. An used herein, the term a “D-amino acid structure” (such as a “D-leucine structure”, a “D-phenylalanine structure” or a “D-valine structure”, is intended to include the D-amino acid, as well as analogues, derivatives and mimetics of the D-amino acid that maintain the functional activity of the compound (discussed further below). For example, the term “D-phenylalanine structure” is intended to include D-phenylalanine as well as D-pyridylalanine and D-homophenylalanine. The term “D-leucine structure” is intended to include D-leucine, as well as substitution with D-valine or other natural or non-natural amino acid having an aliphatic side chain, such as D-norleucine. The term “D-valine structure” is intended to include D-valine, as well as substitution with D-leucine or other natural or non-natural amino acid having an aliphatic side chain.

In other embodiments, the peptidic structure of the β-amyloid peptide derivative comprises at least two D-amino acid structures independently selected from the group consisting of a D-leucine structure, a D-phenylalanine structure, a D-valine structure, a D-alanine structure, a D-tyrosine structure and a D-iodotyrosine structure. In another embodiment, the peptidic structure is comprised of at least three D-amino acid structures independently selected from the group consisting of a D-leucine structure, a D-phenylalanine structure and a D-valine structure. In yet another embodiment, the peptidic structure is comprised of at least three D-amino acid structures independently selected from the group consisting of a D-leucine structure, a D-phenylalanine structure, a D-valine structure, a D-alanine structure, a D-tyrosine structure and a D-iodotyrosine structure. In yet another embodiment, the peptidic structure comprises at least four D-amino acid structures independently selected from the group consisting of a D-leucine structure, a D-phenylalanine structure and a D-valine structure. In yet another embodiment, the peptidic structure is comprised of at least four D-amino acid structures independently selected from the group consisting of a D-leucine structure, a D-phenylalanine structure and a D-valine structure. In a preferred embodiment, the peptidic structure includes a D-amino acid dipeptide selected from the group consisting of D-Phe-D-Phe, D-Phe-D-Tyr, D-Tyr-D-Phe, D-Phe-D-Iodo Tyr and D-Iodo Tyr-D-Phe.

In one embodiment, the invention provides a β-amyloid peptide derivative comprising a formula (I):

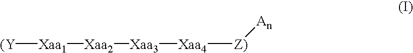

wherein

Xaa₁, Xaa₂, Xaa₃ and Xaa₄ are each D-amino acid structures and at least two of Xaa₁, Xaa₂, Xaa₃ and Xaa₄ are, independently, selected from the group consisting of a D-leucine structure, a D-phenylalanine structure and a D-valine structure;

Y, which may or may not be present, is a structure having the formula (Xaa)ₐ, wherein Xaa is any D-amino acid structure and a is an integer from 1 to 15;

Z, which may or may not be present, is a structure having the formula (Xaa)_{b}, wherein Xaa is any D-amino acid structure and b is an integer from 1 to 15;

A, which may or may not be present, is a modifying group attached directly or indirectly to the β-amyloid peptide derivative; and

n is an integer from 1 to 15;

wherein Xaa₁, Xaa₂, Xaa₃, Xaa₄, Y, Z, A and n are selected such that the β-amyloid peptide derivative inhibits P-glycoprotein and/or cytochrome P450 function and/or binds to natural β-amyloid peptides or modulates the aggregation or inhibits the neurotoxicity of natural β-amyloid peptides when contacted with the natural β-amyloid peptides.

In a subembodiment of this formula, a fifth amino acid residue, Xaa₅, is specified C-terminal to Xaa₄ and Z, which may or may not be present, is a structure having the formula (Xaa)_{b}, wherein Xaa is any D-amino acid structure and b is an integer from 1 to 14. Accordingly, the invention provides a β-amyloid peptide derivative comprising a formula (II):

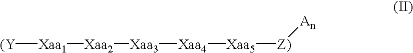

wherein b is an integer from 1 to 14.

In a preferred embodiment, Xaa₁, Xaa₂, Xaa₃, Xaa₄ of formula (I) are selected based on the sequence of Aβ₁₇₋₂₀, or acceptable substitutions thereof. Accordingly, in preferred embodiments, Xaa₁ is a D-alanine structure or a D-leucine structure, Xaa₂ is a D-valine structure, Xaa₃ is a D-phenylalanine structure, a D-tyrosine structure or a D-iodotyrosine structure and Xaa₄ is a D-phenylalanine structure, a D-tyrosine structure or a D-iodotyrosine structure.

In another preferred embodiment, Xaa₁, Xaa₂, Xaa₃, Xaa₄ and Xaa₅ of formula (II) are selected based on the sequence of Aβ₁₇₋₂₁, or acceptable substitutions thereof. Accordingly, in preferred embodiments, Xaa₁ is a D-alanine structure or a D-leucine structure, Xaa₂ is a D-valine structure, Xaa₃ is a D-phenylalanine structure, a D-tyrosine structure or a D-iodotyrosine structure, Xaa₄ is a D-phenylalanine structure, a D-tyrosine structure or a D-iodotyrosine structure, and Xaa₅ is a D-alanine structure or a D-leucine structure.

In another preferred embodiment, Xaa₁, Xaa₂, Xaa₃ and Xaa₄ of formula (I) are selected based on the retro-inverso isomer of Aβ₁₇₋₂₀, or acceptable substitutions thereof. Accordingly, in preferred embodiments, Xaa₁ is a D-alanine structure, a D-leucine structure or a D-phenylalanine structure, Xaa₂ is a D-phenylalanine structure, a D-tyrosine structure or a D-iodotyrosine structure, Xaa₃ is a D-phenylalanine structure, a D-tyrosine structure or a D-iodotyrosine structure and Xaa₄ is a D-valine structure or a D-leucine structure.

In another preferred embodiment, Xaa₁, Xaa₂, Xaa₃, Xaa₄ and Xaa₅ of formula (II) are selected based on the retroinverso isomer of Aβ₁₇₋₂₁, or acceptable substitutions thereof. Accordingly, in preferred embodiments, Xaa₁ is a D-alanine structure, a D-leucine structure or a D-phenylalanine structure, Xaa₂ is a D-phenylalanine structure, a D-tyrosine structure or a D-iodotyrosine structure, Xaa₃ is a D-phenylalanine structure, a D-tyrosine structure or a D-iodotyrosine structure, Xaa₄ is a D-valine structure or a D-leucine structure and Xaa₅ is a D-leucine structure.

In the β-amyloid peptide derivatives of the invention having the formula (I) or (II) shown above, an optional modifying group (“A”) is attached directly or indirectly to the peptidic structure of the β-amyloid peptide derivative. As used herein, the term “modulating group” and “modifying group” are used interchangeably to describe a chemical group directly or indirectly attached to a peptidic structure. For example, a modifying group(s) can be directly attached by covalent coupling to the peptidic structure or a modifying group(s) can be attached indirectly by a stable non-covalent association. In one embodiment of the invention, a modifying group is attached to the amino-terminus of the peptidic structure of the β-amyloid peptide derivative. Alternatively, in another embodiment of the invention, a modifying group is attached to the carboxy-terminus of the peptidic structure of the β-amyloid peptide derivative. In yet another embodiment, a modulating group(s) is attached to the side chain of at least one amino acid residue of the peptidic structure of the β-amyloid peptide derivative (e.g., through the epsilon amino group of a lysyl residue(s), through the carboxyl group of an aspartic acid residue(s) or a glutamic acid residue(s), through a hydroxy group of a tyrosyl residue(s), a serine residue(s) or a threonine residue(s) or other suitable reactive group on an amino acid side chain).

If a modifying group(s) is present, the modifying group is selected such that the β-amyloid peptide derivative inhibits P-glycoprotein and/or cytochrome P450 function, and/or aggregation of natural β-amyloid peptides when contacted with the natural β-amyloid peptides. Accordingly, since the β-AP peptide of the β-amyloid peptide derivative is modified from its natural state, the modifying group “A” as used herein is not intended to include hydrogen. In a β-amyloid peptide derivative of the invention, a single modifying group may be attached to the peptidic structure or multiple modifying groups may be attached to the peptidic structure. The number of modifying groups is selected such that the β-amyloid peptide derivative inhibits P-glycoprotein and/or cytochrome P450 function and/or aggregation of natural β-amyloid peptides when contacted with the natural β-amyloid peptides. However, n preferably is an integer between 1 and 60, more preferably between 1 and 30 and even more preferably between 1 and 10 or 1 and 5. In a preferred embodiment, A is an amino-terminal modifying group comprising a cyclic, heterocyclic, polycyclic or branched alkyl group and n=1. In another preferred embodiment, A is carboxy-terminally modifying group comprising an amide group, an alkyl amide group, an aryl amide group or a hydroxy group, and n=1. Suitable modifying groups are described further in subsections II and III below.

In another embodiment, the invention provides a β-amyloid peptide derivative comprising a formula (III):

A-(Y)-Xaa₁-Xaa₂-Xaa₃-Xaa₄-(Z)-B (III)

wherein

Xaa₁, Xaa₂, Xaa₃ and Xaa₄ are each D-amino acid structures and at least two of Xaa₁, Xaa₂, Xaa₃ and Xaa₄ are, independently, selected from the group consisting of a D-leucine structure, a D-phenylalanine structure and a D-valine structure;

Y, which may or may not be present, is a peptidic structure having the formula (Xaa)ₐ, wherein Xaa is any amino acid structure and a is an integer from 1 to 15;

Z, which may or may not be present, is a peptidic structure having the formula (Xaa)_{b}, wherein Xaa is any amino acid structure and b is an integer from 1 to 15; and

A, which may or may not be present, is a modifying group attached directly or indirectly to the amino terminus of the β-amyloid peptide derivative; and

B, which may or may not be present, is a modifying group attached directly or indirectly to the carboxy terminus of the β amyloid peptide derivative;

Xaa₁, Xaa₂, Xaa₃, Xaa₄, Y, Z, A and B being selected such that the β-amyloid peptide derivative binds to P-glycoprotein and/or cytochrome P450 and inhibits its function and/or binds to natural β-amyloid peptides or modulates the aggregation or inhibits the neurotoxicity of natural β-amyloid peptides when contacted with the natural β-amyloid peptides.

In a subembodiment of formula (III), a fifth amino acid residue, Xaa₅, is specified C-terminal to Xaa₄ and Z, which may or may not be present, is a structure having the formula (Xaa)_{b}, wherein Xaa is any D-amino acid structure and b is an integer from 1 to 14. Accordingly, the invention provides a β-amyloid peptide derivative comprising a formula (IV):

A-(Y)-Xaa₁-Xaa₂-Xaa₃-Xaa₄-Xaa₅-(Z)-B (IV)

wherein b is an integer from 1 to 14.

In a preferred embodiment, Xaa₁, Xaa₂, Xaa₃, Xaa₄ of formula (III) are selected based on the sequence of Aβ₁₇₋₂₀, or acceptable substitutions thereof. Accordingly, in preferred embodiments, Xaa₁ is a D-alanine structure or a D-leucine structure, Xaa₂ is a D-valine structure, Xaa₃ is a D-phenylalanine structure, a D-tyrosine structure or a D-iodotyrosine structure and Xaa₄ is a D-phenylalanine structure, a D-tyrosine structure or a D-iodotyrosine structure.

In another preferred embodiment, Xaa₁, Xaa₂, Xaa₃, Xaa₄ and Xaa₅ of formula (IV) are selected based on the sequence of Aβ₁₇₋₂₁, or acceptable substitutions thereof. Accordingly, in preferred embodiments, Xaa₁ is a D-alanine structure or a D-leucine structure, Xaa₂ is a D-valine structure, Xaa₃ is a D-phenylalanine structure, a D-tyrosine structure or a D-iodotyrosine structure, Xaa₄ is a D-phenylalanine structure, a D-tyrosine structure or a D-iodotyrosine structure, and Xaa₅ is a D-alanine structure or a D-leucine structure.

In another preferred embodiment, Xaa₁, Xaa₂, Xaa₃ and Xaa₄ of formula (III) are selected based on the retro-inverso isomer of Aβ₁₇₋₂₀, or acceptable substitutions thereof. Accordingly, in preferred embodiments, Xaa₁ is a D-alanine structure, a D-leucine structure or a D-phenylalanine structure, Xaa₂ is a D-phenylalanine structure, a D-tyrosine structure or a D-iodotyrosine structure, Xaa₃ is a D-phenylalanine structure, a D-tyrosine structure or a D-iodotyrosine structure and Xaa₄ is a D-valine structure or a D-leucine structure.

In another preferred embodiment, Xaa₁, Xaa₂, Xaa₃, Xaa₄ and Xaa₅ of formula (IV) are selected based on the retroinverso isomer of Aβ₁₇₋₂₁, or acceptable substitutions thereof. Accordingly, in preferred embodiments, Xaa₁ is a D-alanine structure, a D-leucine structure or a D-phenylalanine structure, Xaa₂ is a D-phenylalanine structure, a D-tyrosine structure or a D-iodotyrosine structure, Xaa₃ is a D-phenylalanine structure, a D-tyrosine structure or a D-iodotyrosine structure, Xaa₄ is a D-valine structure or a D-leucine structure and Xaa₅ is a D-leucine structure.

In one embodiment of the β-amyloid peptide derivatives of formulas (III) and/or (IV), A is present and comprises a cyclic, heterocyclic, polycyclic or branched alkyl group. In another embodiment of the β-amyloid peptide derivatives of formulas (III) and/or (IV), B is present and comprises an amide group, an alkyl amide group, an aryl amide group or a hydroxy group. In yet another embodiment of the β-amyloid peptide derivatives of formulas (III) and/or (IV), both A and B are present.

In preferred specific embodiments, the β-amyloid peptide derivative used in the methods of the invention is one of the β-amyloid peptide derivatives shown in Table I, below.

TABLE I

| | | |
|---|---|---|
| D-Leu-D-Val-D-Phe-D-Phe | (SEQ ID NO:1) | |
| D-Leu-D-Val-D-Phe-phenethylamide | (SEQ ID NO:3) | |
| D-Leu-D-Val-D-Tyr-D-Phe | (SEQ ID NO:4) | |
| D-Leu-D-Val-D-IodoTyr-D-Phe | (SEQ ID NO:5) | |
| D-Leu-D-Val-D-Phe-D-Tyr | (SEQ ID NO:6) | |
| D-Leu-D-Val-D-Phe-D-IodoTyr | (SEQ ID NO:7) | |
| D-Leu-D-Val-D-Phe-D-Ala | (SEQ ID NO:8) | |
| D-Leu-D-Val-D-Phe-D-Phe-D-Ala | (SEQ ID NO:2) | |
| D-Ala-D-Val-D-Phe-D-Phe-D-Leu | (SEQ ID NO:9) | |
| D-Leu-D-Val-D-Tyr-D-Phe-D-Ala | (SEQ ID NO:10) | |
| D-Leu-D-Val-D-IodoTyr-D-Phe-D-Ala | (SEQ ID NO:11) | |
| D-Leu-D-Val-D-Phe-D-Tyr-D-Ala | (SEQ ID NO:12) | |
| D-Leu-D-Val-D-Phe-D-IodoTyr-D-Ala | (SEQ ID NO:13) | |
| D-Phe-D-Phe-D-Val-D-Leu | (SEQ ID NO:14) | |
| D-Ala-D-Phe-D-Phe-D-Val | (SEQ ID NO:15) | |
| D-Ala-D-Phe-D-Phe-D-Val-D-Leu | (SEQ ID NO:16) | |
| D-Ala-D-Phe-D-Phe-D-Leu-D-Leu | (SEQ ID NO:17) | |
| D-Leu-D-Phe-D-Phe-D-Val-D-Leu | (SEQ ID NO:18) | PPI-578 |
| D-Phe-D-Phe-D-Phe-D-Val-D-Leu | (SEQ ID NO:19) | |
| D-Phe-D-Phe-D-Phe-D-Leu-D-Val | (SEQ ID NO:20) | |
| D-Phe-D-Phe-D-Phe-D-Phe-D-Leu | (SEQ ID NO:21) | |
| D-Ala-D-Phe-D-Phe-D-Phe-D-Leu | (SEQ ID NO:22) | |
| N-methyl-(D-Leu-D-Val-D-Phe-D-Phe-D-Leu)-NH₂ | | PPI-1019 |
| 4-Hydroxybenzoyl-D-Leu-D-Val-D-Phe-D-Phe-D-Ala-NH₂ | | PPI-558 |
| H-D-Leu-D-Val-D-Phe-D-Phe-D-Val-NH2 | | PPI-657 |
| H-D-Leu-D-Val-D-Phe-D-Phe-D-Leu-NH2 | | PPI-655 |

Any of the aforementioned specific peptidic structures can be amino-terminally and/or carboxy-terminally modified and described further in subsections II and/or III below.

Particularly preferred β-amyloid peptide derivatives comprise D-amino acid peptide amides designed based on the retro-inverso isomer of Aβ₁₇₋₂₁, or acceptable substitutions thereof, including β-amyloid peptide derivatives selected from the group consisting of D-Ala-D-Phe-D-Phe-D-Val-D-Leu-amide (SEQ ID NO:16; C-terminal amide), D-Ala-D-Phe-D-Phe-D-Leu-D-Leu-amide (SEQ ID NO:17; C-terminal amide), D-Leu-D-Phe-D-Phe-D-Val-D-Leu-amide (SEQ ID NO:18; C-terminal amide), D-Phe-D-Phe-D-Phe-D-Val-D-Leu-amide (SEQ ID NO:19; C-terminal amide), D-Phe-D-Phe-D-Phe-D-Leu-D-Val-amide (SEQ ID NO:20; C-terminal amide), D-Phe-D-Phe-D-Phe-D-Phe-D-Leu-amide (SEQ ID NO:21; C-terminal amide) and D-Ala-D-Phe-D-Phe-D-Phe-D-Leu-amide (SEQ ID NO:22; C-terminal amide).

The D-amino acid peptidic structures of the β-amyloid peptide derivatives of the invention are further intended to include other peptide modifications, including analogues, derivatives and mimetics, that retain the ability of the β-amyloid peptide derivative to inhibit P-glycoprotein and/or cytochrome P450 function and/or alter natural β-AP aggregation as described herein. For example, a D-amino acid peptidic structure of a β-amyloid peptide derivative of the invention may be further modified to increase its stability, bioavailability, solubility and the like. The terms “analogue”, “derivative” and “mimetic” as used herein are intended to include molecules which mimic the chemical structure of a D-peptidic structure and retain the functional properties of the D-peptidic structure. Approaches to designing peptide analogs, derivatives and mimetics are known in the art. For example, see Farmer, P. S. in Drug Design (E. J. Ariens, ed.) Academic Press, New York, 1980, vol. 10, pp. 119-143; Ball. J. B. and Alewood, P. F. (1990) J. Mol. Recognition 3:55; Morgan, B. A. and Gainor, J. A. (1989) Ann. Rep. Med. Chem. 24:243; and Freidinger, R. M. (1989) Trends Pharmacol. Sci. 10:270. See also Sawyer, T. K. (1995) “Peptidomimetic Design and Chemical Approaches to Peptide Metabolism” in Taylor, M. D. and Amidon, G. L. (eds.) Peptide-Based Drug Design: Controlling Transport and Metabolism, Chapter 17; Smith, A. B. 3rd, et al. (1995) J. Am. Chem. Soc. 117:11113-11123; Smith, A. B. 3rd, etal. (1994) J. Am. Chem. Soc. 116:9947-9962; and Hirschman, R., et al. (1993) J. Am. Chem. Soc. 115:12550-12568.

As used herein, a “derivative” of a compound X (e.g., a peptide or amino acid) refers to a form of X in which one or more reaction groups on the compound have been derivatized with a substituent group. Examples of peptide derivatives include peptides in which an amino acid side chain, the peptide backbone, or the amino- or carboxy-terminus has been derivatized (e.g, peptidic compounds with methylated amide linkages). As used herein an “analogue” of a compound X refers to a compound which retains chemical structures of X necessary for functional activity of X yet which also contains certain chemical structures which differ from X. An examples of an analogue of a naturally-occurring peptide is a peptide which includes one or more non-naturally-occurring amino acids. As used herein, a “mimetic” of a compound X refers to a compound in which chemical structures of X necessary for functional activity of X have been replaced with other chemical structures which mimic the conformation of X. Examples of peptidomimetics include peptidic compounds in which the peptide backbone is substituted with one or more benzodiazepine molecules (see e.g., James, G. L. et al. (1993) Science 260:1937-1942).

Analogues of the β-amyloid peptide derivatives of the invention are intended to include β-amyloid peptide derivatives in which one or more D-amino acids of the peptidic structure are substituted with a homologous amino acid such that the properties of the original β-amyloid peptide derivative are maintained. Preferably conservative amino acid substitutions are made at one or more amino acid residues. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g, aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine, tryptophan, histidine). Non-limiting examples of homologous substitutions that can be made in the peptidic structures of the β-amyloid peptide derivatives of the invention include substitution of D-phenylalanine with D-tyrosine, D-pyridylalanine or D-homophenylalanine, substitution of D-leucine with D-valine or other natural or non-natural amino acid having an aliphatic side chain and/or substitution of D-valine with D-leucine or other natural or non-natural amino acid having an aliphatic side chain. Preferred examples of homologous amino acids which can be used include cyclohexyl-phenylalanine, pentafluoro-phenylalanine, and parafluoro-phenylalanine.

The term mimetic, and in particular, peptidomimetic, is intended to include isosteres. The term “isostere” as used herein is intended to include a chemical structure that can be substituted for a second chemical structure because the steric conformation of the first structure fits a binding site specific for the second structure. The term specifically includes peptide back-bone modifications (i.e., amide bond mimetics) well known to those skilled in the art. Such modifications include modifications of the amide nitrogen, the α-carbon, amide carbonyl, complete replacement of the amide bond, extensions, deletions or backbone crosslinks. Several peptide backbone modifications are known, including Ψ[CH₂S], Ψ[CH₂NH], Ψ[CSNH₂], Ψ[NHCO], Ψ[COCH₂], and Ψ[(E) or (Z) CH═CH]. In the nomenclature used above, Ψ indicates the absence of an amide bond. The structure that replaces the amide group is specified within the brackets.

Other possible modifications include an N-alkyl (or aryl) substitution (Ψ[CONR]), or backbone crosslinking to construct lactams and other cyclic structures. Other derivatives of the β-amyloid peptide derivatives of the invention include C-terminal hydroxymethyl derivatives, O-modified derivatives (e.g., C-terminal hydroxymethyl benzyl ether), N-terminally modified derivatives including substituted amides such as alkylamides and hydrazides and compounds in which a C-terminal phenylalanine residue is replaced with a phenethylamide analogue (e.g., Val-Phe-phenethylamide as an analogue of the tripeptide Val-Phe-Phe).

The β-amyloid peptide derivatives of the invention can be incorporated into pharmaceutical compositions, e.g., compositions which also contain P-glycoprotein inhibitors and/or cytochrome P450 inhibitors, and can be used in methods for increasing the bioavailability of a drug, e.g., the bioavailability of a drug to the brain and/or the oral bioavailability of a drug.

III. Modifying Groups

In certain embodiments of the β-amyloid peptide derivatives of the invention, a hydrophobic peptidic structure (such as an Aβ derived peptide, or an Aβ aggregation core domain, or an amino acid sequence corresponding to a rearranged Aβ aggregation core domain) is coupled directly or indirectly to at least one modifying group (abbreviated as MG). The term “modifying group” is intended to include structures that are directly attached to the hydrophobic peptidic structure (e.g., by covalent coupling), as well as those that are indirectly attached to the peptidic structure (e.g., by a stable non-covalent association or by covalent coupling to additional amino acid residues, or mimetics, analogues or derivatives thereof, which may flank the Aβ-derived D-amino acid peptidic structure). For example, the modifying group can be coupled to the amino-terminus or carboxy-terminus of an Aβ-derived D-amino acid peptidic structure, or to a peptidic or peptidomimetic region flanking the core domain. Alternatively, the modifying group can be coupled to a side chain of at least one D-amino acid residue of an Aβ-derived D-amino acid peptidic structure, or to a peptidic or peptidomimetic region flanking the core domain (e.g., through the epsilon amino group of a lysyl residue(s), through the carboxyl group of an aspartic acid residue(s) or a glutamic acid residue(s), through a hydroxy group of a tyrosyl residue(s), a serine residue(s) or a threonine residue(s) or other suitable reactive group on an amino acid side chain). Modifying groups covalently coupled to the D-amino acid peptidic structure can be attached by means and using methods well known in the art for linking chemical structures, including, for example, amide, alkylamino, carbamate, urea or ester bonds.

The term “modifying group” is intended to include groups that are not naturally coupled to natural Aβ peptides in their native form. Accordingly, the term “modifying group” is not intended to include hydrogen. The modifying group(s) is selected such that the β-amyloid peptide derivative inhibits P-glycoprotein and/or cytochrome P450 function and/or alters, and preferably inhibits, aggregation of natural β-amyloid peptides when contacted with the natural β-amyloid peptides or inhibits the neurotoxicity of natural β-amyloid peptides when contacted with the natural β-amyloid peptides. Although not intending to be limited by mechanism, in embodiments where the β-amyloid peptide derivative comprises a modifying group(s), the modifying group(s) is thought to function as a key pharmacophore that enhances the ability of the β-amyloid peptide derivative to inhibit P-glycoprotein and/or cytochrome P450 function and/or to disrupt Aβ polymerization.

In a preferred embodiment, the modifying group(s) comprises an alkyl, such as a C1-C6 lower alkyl group, e.g., methyl, ethyl, or propyl group; or a cyclic, heterocyclic, polycyclic or branched alkyl group. The term “cyclic group”, as used herein, is intended to include cyclic saturated or unsaturated (i. e., aromatic) group having from about 3 to 10, preferably about 4 to 8, and more preferably about 5 to 7, carbon atoms. Exemplary cyclic groups include cyclopropyl, cyclobutyl, cyclopentyl, cyclohexyl, and cyclooctyl. Cyclic groups may be unsubstituted or substituted at one or more ring positions. Thus, a cyclic group may be substituted with, e.g., halogens, alkyls, cycloalkyls, alkenyls, alkynyls, aryls, heterocycles, hydroxyls, aminos, nitros, thiols amines, imines, amides, phosphonates, phosphines, carbonyls, carboxyls, silyls, ethers, thioethers, sulfonyls, sulfonates, selenoethers, ketones, aldehydes, esters, —CF₃, —CN, or the like.

The term “heterocyclic group” is intended to include cyclic saturated or unsaturated (ie., aromatic) group having from about 3 to 10, preferably about 4 to 8, and more preferably about 5 to 7, carbon atoms, wherein the ring structure includes about one to four heteroatoms. Heterocyclic groups include pyrrolidine, oxolane, thiolane, imidazole, oxazole, piperidine, piperazine, morpholine and pyridine. The heterocyclic ring can be substituted at one or more positions with such substituents as, for example, halogens, alkyls, cycloalkyls, alkenyls, alkynyls, aryls, other heterocycles, hydroxyl, amino, nitro, thiol, amines, imines, amides, phosphonates, phosphines, carbonyls, carboxyls, silyls, ethers, thioethers, sulfonyls, selenoethers, ketones, aldehydes, esters, —CF₃, —CN, or the like. Heterocycles may also be bridged or fused to other cyclic groups as described below.

The term “polycyclic group” as used herein is intended to refer to two or more saturated or unsaturated (i.e., aromatic) cyclic rings in which two or more carbons are common to two adjoining rings, e.g., the rings are “fused rings”. Rings that are joined through non-adjacent atoms are termed “bridged” rings. Each of the rings of the polycyclic group can be substituted with such substituents as described above, as for example, halogens, alkyls, cycloalkyls, alkenyls, alkynyls, hydroxyl, amino, nitro, thiol, amines, imines, amides, phosphonates, phosphines, carbonyls, carboxyls, silyls, ethers, thioethers, sulfonyls, selenoethers, ketones, aldehydes, esters, —CF₃, —CN, or the like.

A preferred polycyclic group is a group containing a cis-decalin structure. Although not intending to be limited by mechanism, it is thought that the “bent” conformation conferred on a modifying group by the presence of a cis-decalin structure contributes to the efficacy of the modifying group in disrupting Aβ polymerization. Accordingly, other structures which mimic the “bent” configuration of the cis-decalin structure can also be used as modifying groups. An example of a cis-decalin containing structure that can be used as a modifying group is a cholanoyl structure, such as a cholyl group. For example, a modulator compound can be modified at its amino terminus with a cholyl group by reacting the aggregation core domain with cholic acid, a bile acid. Moreover, a modulator compound can be modified at its carboxy terminus with a cholyl group according to methods known in the art (see e.g., Wess, G. et al. (1993) Tetrahedron Letters, 34:817-822; Wess, G. et al. (1992) Tetrahedron Letters 33:195-198; and Kramer, W. et al. (1992) J. Biol. Chem. 267:18598-18604). Cholyl derivatives and analogues can also be used as modifying groups. For example, a preferred cholyl derivative is Aic (3-(O-aminoethyl-iso)-cholyl), which has a free amino group that can be used to further modify the modulator compound (e.g., a chelation group for ^{99m}Tc can be introduced through the free amino group of Aic). As used herein, the term “cholanoyl structure” is intended to include the cholyl group and derivatives and analogues thereof, in particular those which retain a four-ring cis-decalin configuration. Examples of cholanoyl structures include groups derived from other bile acids, such as deoxycholic acid, lithocholic acid, ursodeoxycholic acid, chenodeoxycholic acid and hyodeoxycholic acid, as well as other related structures such as cholanic acid, bufalin and resibufogenin (although the latter two compounds are not preferred for use as a modifying group). Another example of a cis-decalin containing compound is 5β-cholestan-3β-ol (the cis-decalin isomer of (+)-dihydrocholesterol). For further description of bile acid and steroid structure and nomenclature, see Nes, W. R. and McKean, M. L. Biochemistry of Steroids and Other Isopentanoids, University Park Press, Baltimore, Md, Chapter 2.

In addition to cis-decalin containing groups, other polycyclic groups may be used as modifying groups. For example, modifying groups derived from steroids or β-lactams may be suitable modifying groups. In one embodiment, the modifying group is a “biotinyl structure”, which includes biotinyl groups and analogues and derivatives thereof (such as a 2-iminobiotinyl group). In another embodiment, the modifying group can comprise a “fluorescein-containing group”, such as a group derived from reacting an Aβ-derived peptidic structure with 5-(and 6-)-carboxyfluorescein, succinimidyl ester or fluorescein isothiocyanate. In various other embodiments, the modifying group(s) can comprise an N-acetylneuraminyl group, a trans-4-cotininecarboxyl group, a 2-imino-1-imidazolidineacetyl group, an (S)-(−)-indoline-2-carboxyl group, a (−)-menthoxyacetyl group, a 2-norbornaneacetyl group, a γ-oxo-5-acenaphthenebutyryl, a (−)-2-oxo-4-thiazolidinecarboxyl group, a tetrahydro-3-furoyl group, a 2-iminobiotinyl group, a diethylenetriaminepentaacetyl group, a 4-morpholinecarbonyl group, a 2-thiopheneacetyl group or a 2-thiophenesulfonyl group.

In addition to the cyclic, heterocyclic and polycyclic groups discussed above, other types of modifying groups can be used in a β-amyloid peptide derivative of the invention. For example, hydrophobic groups and branched alkyl groups may be suitable modifying groups. Examples include acetyl groups, phenylacetyl groups, phenylacetyl groups, diphenylacetyl groups, triphenylacetyl groups, isobutanoyl groups, 4-methylvaleryl groups, trans-cinnamoyl groups, butanoyl groups and 1-adamantanecarbonyl groups.

Yet another type of modifying group is a compound that contains a non-natural amino acid that acts as a beta-turn mimetic, such as a dibenzofuran-based amino acid described in Tsang, K. Y. et al. (1994) J. Am. Chem. Soc. 116:3988-4005; Diaz, H and Kelly, J. W. (1991) Tetrahedron Letters 41:5725-5728; and Diaz. H et al. (1992) J. Am. Chem. Soc. 114:8316-8318. An example of such a modifying group is a peptide-aminoethyldibenzofuranyl-proprionic acid (Adp) group (e.g., DDIIL-Adp) (SEQ ID NO:23). This type of modifying group further can comprise one or more N-methyl peptide bonds to introduce additional steric hindrance to the aggregation of natural β-AP when compounds of this type interact with natural β-AP.

Non-limiting examples of suitable modifying groups, with their corresponding modifying reagents, are listed below:

| Modifying Group | Modifying Reagent |
|---|---|
| Methyl- | Methylamine, Fmoc-D-[Me]-Leu- |
| | OH, methylamine and a |
| | bromoacetylpeptide |
| Ethyl- | Ethylamine, acetaldehyde and sodium |
| | cyanoborohydride, ethylamine and a |
| | bromoacetylpeptide |
| Propyl- | Propylamine, propionaldehyde and |
| | sodium cyanoborohydride, propyl- |
| | amine and a bromoacetylpeptide |
| Isopropyl- | Isopropylamine, isopropylamine and |
| | a bromoacetylpeptide |
| Piperidine- | Piperidine and a bromoacetylpeptide |
| Acetyl- | Acetic anhydride, acetic acid |
| Dimethyl- | Methylamine, formaldehyde and |
| | sodium cyanoborohydride |
| Diethyl- | Acetaldehyde and sodium |
| | cyanoborohydride |
| Cholyl- | Cholic acid |
| Lithocholyl- | Lithocholic acid |
| Hyodeoxycholyl- | Hyodeoxycholic acid |
| Chenodeoxycholyl- | Chenodeoxycholic acid |
| Ursodeoxycholyl- | Ursodeoxycholic acid |
| 3-Hydroxycinnamoyl- | 3-Hydroxycinnamic acid |
| 4-Hydroxycinnamoyl- | 4-Hydroxycinnamic acid |
| 2-Hydroxycinnamoyl- | 2-Hydroxycinnamic acid |
| 3-Hydroxy-4-methoxycinnamoyl- | 3-Hydroxy-4-methoxycinnamic acid |
| 4-Hydroxy-3-methoxycinnamoyl- | 4-Hydroxy-3-methoxycinnamic acid |
| 2-Carboxycinnamoyl- | 2-Carboxycinnamic acid |
| 3-Formylbenzoyl | 3-Carboxybenzaldehyde |
| 4-Formylbenzoyl | 4-Carboxybenzaldehyde |
| 3,4,-Dihydroxyhydrocinnamoyl- | 3,4,-Dihydroxyhydrocinnamic acid |
| 3,7-Dihydroxy-2-napthoyl- | 3,7-Dihydroxy-2-naphthoic acid |
| 4-Formylcinnamoyl- | 4-Formylcinnamic acid |
| 2-Formylphenoxyacetyl- | 2-Formylphenoxyacetic acid |
| 8-Formyl-1-napthoyl | 1,8-napthaldehydic acid |
| 4-(hydroxymethyl)benzoyl- | 4-(hydroxymethyl)benzoic acid |
| 4-Hydroxyphenylacetyl- | 4-Hydroxyphenylacetic acid |
| 3-Hydroxybenzoyl- | 3-Hydroxybenzoic acid |
| 4-Hydroxybenzoyl- | 4-Hydroxybenzoic acid |
| 5-Hydantoinacetyl- | 5-Hydantoinacetic acid |
| L-Hydroorotyl- | L-Hydroorotic acid |
| 4-Methylvaleryl- | 4-Methylvaleric acid |
| 2,4-Dihydroxybenzoyl- | 2,4-Dihydroxybenzoic acid |
| 3,4-Dihydroxycinnamoyl- | 3,4-Dihydroxycinnamic acid |
| 3,5-Dihydroxy-2-naphthoyl- | 3,5-Dihydroxy-2-naphthoic acid |
| 3-Benzoylpropanoyl- | 3-Benzoylpropanoic acid |
| trans-Cinnamoyl- | trans-Cinnamic acid |
| Phenylacetyl- | Phenylacetic acid |
| Diphenylacetyl- | Diphenylacetic acid |
| Triphenylacetyl- | Triphenylacetic acid |
| 2-Hydroxyphenylacetyl- | 2-Hydroxyphenylacetic acid |
| 3-Hydroxyphenylacetyl- | 3-Hydroxyphenylacetic acid |
| 4-Hydroxyphenylacetyl- | 4-Hydroxyphenylacetic acid |
| (±)-Mandelyl- | (±)-Mandelic acid |
| (±)-2,4-Dihydroxy-3,3- | (±)-Pantolactone |
| dimethylbutanoyl | |
| Butanoyl- | Butanoic anhydride |
| Isobutanoyl- | Isobutanoic anhydride |
| Hexanoyl- | Hexanoic anhydride |
| Propionyl- | Propionic anhydride |
| 3-Hydroxybutyroyl | β-Butyrolactone |
| 4-Hydroxybutyroyl | γ-Butyrolactone |
| 3-Hydroxypropionoyl | β-Propiolactone |
| 2,4-Dihydroxybutyroyl | α-Hydroxy-β-Butyrolactone |
| 1-Adamantanecarbonyl- | 1-Adamantanecarbonic acid |
| Glycolyl- | Glycolic acid |
| DL-3-(4-hydroxyphenyl)lactyl- | DL-3-(4-hydroxyphenyl)lactic acid |
| 3-(2-Hydroxyphenyl)propionyl- | 3-(2-Hydroxyphenyl)propionic acid |
| 4-(2-Hydroxyphenyl)propionyl- | 4-(2-Hydroxyphenyl)propionic acid |
| D-3-Phenyllactyl- | D-3-Phenyllactic acid |
| Hydrocinnamoyl- | Hydrocinnamic acid |
| 3-(4-Hydroxyphenyl)propionyl- | 3-(4-Hydroxyphenyl)propionic acid |
| L-3-Phenyllactyl- | L-3-Phenyllactic acid |
| 4-methylvaleryl | 4-methylvaleric acid |
| 3-pyridylacetyl | 3-pyridylacetic acid |
| 4-pyridylacetyl | 4-pyridylacetic acid |
| Isonicotinoyl | |
| 4-quinolinecarboxyl | 4-quinolinecarboxylic acid |
| 1-isoquinolinecarboxyl | 1-isoquinolinecarboxylic acid |
| 3-isoquinolinecarboxyl | 3-isoquinolinecarboxylic acid |

Preferred modifying groups include methyl-containing groups, ethyl-containing groups, propyl-containing groups, and piperidine-containing groups, e.g., a 1-piperidine-acetyl group.

Preferred modifying groups also include cis-decalin-containing groups, biotin-containing groups, fluorescein-containing groups, a diethylene-triaminepentaacetyl group, a (−)-menthoxyacetyl group, an N-acetylneuraminyl group, a phenylacetyl group, a diphenylacetyl group, a triphenylacetyl group, an isobutanoyl group, a 4-methylvaleryl group, a 3-hydroxyphenylacetyl group, a 2-hydroxyphenylacetyl group, a 3,5-dihydroxy-2-naphthoyl group, a 3,4-dihydroxycinnamoyl group, a (±)-mandelyl group, a (±)-mandelyl-(±)-mandelyl group, a glycolyl group, a benzoylpropanoyl group and a 2,4-dihydroxybenzoyl group.

VI. Additional Chemical Modifications of β-amyloid Peptide Derivatives

A β-amyloid peptide derivative of the invention can be further modified to alter the specific properties of the β-amyloid peptide derivative while retaining the ability of the β-amyloid peptide derivative to inhibit P-glycoprotein and/or cytochrome P450 function and/or to alter Aβ aggregation and inhibit Aβ neurotoxicity. For example, in one embodiment, the β-amyloid peptide derivative is further modified to alter a pharmacokinetic property of the β-amyloid peptide derivative, such as in vivo stability or half-life. In another embodiment, the β-amyloid peptide derivative is further modified to label the β-amyloid peptide derivative with a detectable substance. In yet another embodiment, the β-amyloid peptide derivative is further modified to couple the β-amyloid peptide derivative to an additional therapeutic moiety. Schematically, β-amyloid peptide derivative of the invention comprising a D-amino acid Aβ aggregation core domain coupled directly or indirectly to at least one modifying group can be illustrated as MG-ACD, whereas this compound which has been further modified to alter the properties of the β-amyloid peptide derivative can be illustrated as MG-ACD-CM, wherein CM represents an additional chemical modification.

To further chemically modify the β-amyloid peptide derivative, such as to alter the pharmacokinetic properties of the β-amyloid peptide derivative, reactive groups can be derivatized. For example, when the modifying group is attached to the amino-terminal end of the aggregation core domain, the carboxy-terminal end of the β-amyloid peptide derivative can be further modified. Preferred C-terminal modifications include those which reduce the ability of the β-amyloid peptide derivative to act as a substrate for carboxypeptidases. Examples of preferred C-terminal modifiers include an amide group (i.e., a peptide amide), an alkyl or aryl amide group (e.g., an ethylamide group or a phenethylamide group) a hydroxy group (i.e., a peptide alcohol) and various non-natural amino acids, such as D-amino acids and β-alanine. Alternatively, when the modifying group is attached to the carboxy-terminal end of the aggregation core domain, the amino-terminal end of the β-amyloid peptide derivative can be further modified, for example, to reduce the ability of the compound to act as a substrate for aminopeptidases.

A β-amyloid peptide derivative can be further modified to label the compound by reacting the compound with a detectable substance. Suitable detectable substances include various enzymes, prosthetic groups, fluorescent materials, luminescent materials and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include ¹⁴C, ¹²³I, ¹²⁴I, ¹²⁵I, ¹³¹I, ^{99m}Tc, ³⁵S or ³H. In a preferred embodiment, a β-amyloid peptide derivative is radioactively labeled with ¹⁴C, either by incorporation of ¹⁴C into the modifying group or one or more amino acid structures in the β-amyloid peptide derivative. Labeled β-amyloid peptide derivatives can be used to assess the in vivo pharmacokinetics of the β-amyloid peptide derivatives, as well as to detect P-glycoprotein and/or cytochrome P450 binding and/or Aβ aggregation, for example for diagnostic purposes. P-glycoprotein and/or cytochrome P450 binding and/or Aβ aggregation can be detected using a labeled β-amyloid peptide derivative either in vivo or in an in vitro sample derived from a subject.

Preferably, for use as an in vivo diagnostic agent, a β-amyloid peptide derivative of the invention is labeled with radioactive technetium or iodine. Accordingly, in one embodiment, the invention provides a β-amyloid peptide derivative labeled with technetium, preferably ^{99m}Tc. Methods for labeling peptide compounds with technetium are known in the art (see e.g., U.S. Pat. Nos. 5,443,815, 5,225,180 and 5,405,597, all by Dean et al.; Stepniak-Biniakiewicz, D., et al. (1992) J. Med. Chem. 35:274-279; Fritzberg, A. R., et al. (1988) Proc. Natl. Acad. Sci. USA 85:4025-4029; Baidoo, K. E., et al. (1990) Cancer Res. Suppl. 50:799s-803s; and Regan, L. and Smith, C. K. (1995) Science 270:980-982). A modifying group can be chosen that provides a site at which a chelation group for ^{99m}Tc can be introduced, such as the Aic derivative of cholic acid, which has a free amino group. In another embodiment, the invention provides a β-amyloid peptide derivative labeled with radioactive iodine. For example, a phenylalanine residue within the Aβ sequence (such as Phe₁₉ or Phe₂₀) can be substituted with radioactive iodotyrosyl. Any of the various isotopes of radioactive iodine can be incorporated to create a diagnostic agent. Preferably, ¹²³I (half-life=13.2 hours) is used for whole body scintigraphy, ¹²⁴I (half life=4 days) is used for positron emission tomography (PET), ¹²⁵I (half life=60 days) is used for metabolic turnover studies and ¹³¹I (half life=8 days) is used for whole body counting and delayed low resolution imaging studies.

Furthermore, an additional modification of a β-amyloid peptide derivative of the invention can serve to confer an additional therapeutic property on the β-amyloid peptide derivative. That is, the additional chemical modification can comprise an additional functional moiety. For example, a functional moiety which serves to break down or dissolve amyloid plaques can be coupled to the β-amyloid peptide derivative. In this form, the MG-ACD portion of the β-amyloid peptide derivative serves to target the β-amyloid peptide derivative to the P-glycoprotein and/or to the cytochrome P450 and inhibit its function and/or to Aβ peptides and disrupt the polymerization of the Aβ peptides, whereas the additional functional moiety serves to break down or dissolve amyloid plaques after the β-amyloid peptide derivative has been targeted to these sites.

In an alternative chemical modification, a β-amyloid peptide derivative of the invention is prepared in a “prodrug” form, wherein the compound itself does not inhibit P-glycoprotein and/or cytochrome P450 function and/or modulate Aβ aggregation, but rather is capable of being transformed, upon metabolism in vivo, into a β-amyloid peptide derivative as defined herein. For example, in this type of compound, the modulating group can be present in a prodrug form that is capable of being converted upon metabolism into the form of an active modulating group. Such a prodrug form of a modifying group is referred to herein as a “secondary modifying group.” A variety of strategies are known in the art for preparing peptide prodrugs that limit metabolism in order to optimize delivery of the active form of the peptide-based drug (see e.g., Moss, J. (1995) in Peptide-Based Drug Design: Controlling Transport and Metabolism, Taylor, M. D. and Amidon, G. L. (eds), Chapter 18. Additionally strategies have been specifically tailored to achieving CNS delivery based on “sequential metabolism” (see e.g., Bodor, N., et al. (1992) Science 257:1698-1700; Prokai, L., et al. (1994) J. Am. Chem. Soc. 116:2643-2644; Bodor, N. and Prokai, L. (1995) in Peptide-Based Drug Design: Controlling Transport and Metabolism, Taylor, M. D. and Amidon, G. L. (eds), Chapter 14. In one embodiment of a prodrug form of a β-amyloid peptide derivative of the invention, the modifying group comprises an alkyl ester to facilitate blood-brain barrier permeability.

β-amyloid peptide derivatives of the invention can be prepared by standard techniques known in the art. The peptide component of a β-amyloid peptide derivative can be synthesized using standard techniques such as those described in Bodansky, M. Principles of Peptide Synthesis, Springer Verlag, Berlin (1993) and Grant, G. A (ed.). Synthetic Peptides: A User's Guide, W. H. Freeman and Company, New York (1992). Automated peptide synthesizers are commercially available (e.g., Protein Technologies Model PS3; Renin Instruments Model Symphony). Additionally, one or more modulating groups can be attached to the Aβ-derived peptidic component (e.g., an Aβ aggregation core domain) by standard methods, for example using methods for reaction through an amino group (e.g., the alpha-amino group at the amino-terminus of a peptide), a carboxyl group (e.g, at the carboxy terminus of a peptide), a hydroxyl group (e.g, on a tyrosine, serine or threonine residue) or other suitable reactive group on an amino acid side chain (see e.g., Greene, T. W and Wuts, P. G. M. Protective Groups in Organic Synthesis, John Wiley and Sons, Inc., New York (1991). Exemplary syntheses of β-amyloid peptide derivatives are described further in Example 1.

V. Methods For Enhancing the Bioavailability of a β-Amyloid Peptide Derivative to the Brain of a Subject

Another aspect of the invention pertains to a method for enhancing the bioavailability of a β-amyloid peptide derivative to the brain of a subject. The method includes administering to the subject the β-amyloid peptide derivative and a P-glycoprotein inhibitor, thereby enhancing the bioavailability of the β-amyloid peptide derivative to the brain of the subject. Any of the β-amyloid peptide derivatives described herein may be used in the aforementioned methods. In a preferred embodiment, the β-amyloid peptide derivative is PPI-558, PPI-657, PPI-1019, PPI-578, or PPI-655, more preferably, PPI-1019.

P-glycoprotein inhibitors suitable for use in the methods of the invention are known in the art and include antiarrhythmic agents, antibiotics, antifungal agents, calcium channel blockers, chemotherapeutic agents, hormones, antiparasitic agents, local anesthetics, phenothiazines, and tricyclic antidepressants. P-glycoprotein inhibitors are described in, for example, U.S. Pat. Nos. 5,567,592, 5,776,939, and PCT Application No. WO 95/31474, the contents of which are incorporated herein by reference. Preferred P-glycoprotein inhibitors include cyclosporin A and valspodar.

Suitable dosages and routes of administration for the β-amyloid derivative and the P-glycoprotein inhibitor include those described in Example 2 and the figures.

In one embodiment, the method further includes administering a cytochrome P450 inhibitor to the subject. Cytochrome P450 inhibitors suitable for use in the methods of the invention are known in the art and include calcium channel blockers, e.g., Verapamil, Felodipine, or Diltiazem; flavanoids, e.g., Quercetin, Kaempherol, or Benzoflavone; steroid hormones, e.g., cortisol, or progesterone; chemotherapeutic agents; or antidiabetic agents, e.g., Tolbutamide. Cytochrome P450 inhibitors are described in, for example, PCT Application No. WO 95/20980, the contents of which are incorporated herein by reference.

The β-amyloid peptide derivative and the P-glycoprotein inhibitor may be administered simultaneously or at different times. For example, the β-amyloid peptide derivative can be administered every 2, 4, 6, 8, 10, 12, 24, or 48 hours, and the P-glycoprotein inhibitor can be administered every 2, 4, 6, 8, 10, 12, 24, or 48 hours, wherein the time of administration of the β-amyloid peptide derivative and the P-glycoprotein inhibitor may be the same or different. Furthermore, the β-amyloid peptide derivative and the P-glycoprotein inhibitor may be administered in the same pharmaceutical formulation or in different pharmaceutical formulations. Suitable pharmaceutical formulations for the administration of the β-amyloid peptide derivative and the P-glycoprotein inhibitor are described herein.

Yet another aspect of the invention pertains to a method for enhancing the bioavailability of a β-amyloid peptide derivative to the brain of a subject by administering to the subject the β-amyloid peptide derivative and a cytochrome P450 inhibitor, thereby enhancing the bioavailability of the β-amyloid peptide derivative to the brain of the subject.

The β-amyloid peptide derivative and the cytochrome P450 inhibitor may be administered simultaneously or at different times. For example, the β-amyloid peptide derivative can be administered every 2, 4, 6, 8, 10, 12, 24, or 48 hours, and the cytochrome P450 inhibitor can be administered every 2, 4, 6, 8, 10, 12, 24, or 48 hours, wherein the time of administration of the β-amyloid peptide derivative and the cytochrome P450 inhibitor may be the same or different. Furthermore, the β-amyloid peptide derivative and the cytochrome P450 inhibitor may be administered in the same pharmaceutical formulation or in different pharmaceutical formulations. Suitable pharmaceutical formulations for the administration of the β-amyloid peptide derivative and the cytochrome P450 inhibitor are described herein.

In one embodiment, the method further includes administering a P-glycoprotein inhibitor to the subject. The β-amyloid peptide derivative, the P-glycoprotein inhibitor, and the cytochrome P450 inhibitor may be administered simultaneously or at different times. For example, the β-amyloid peptide derivative can be administered every 2, 4, 6, 8, 10, 12, 24, or 48 hours, the P-glycoprotein inhibitor can be administered every 2, 4, 6, 8, 10, 12, 24, or 48 hours, and the cytochrome P450 inhibitor can be administered every 2, 4, 6, 8, 10, 12, 24, or 48 hours, wherein the time of administration of the β-amyloid peptide derivative, the P-glycoprotein inhibitor, and the cytochrome P450 inhibitor may be the same or different. Furthermore, the β-amyloid peptide derivative, the P-glycoprotein inhibitor, and the cytochrome P450 inhibitor may be administered in the same pharmaceutical formulation or in different pharmaceutical formulations. Suitable pharmaceutical formulations for the administration of the β-amyloid peptide derivative, the P-glycoprotein inhibitor, and the cytochrome P450 inhibitor are described herein.

VI. Methods for Treating or Preventing Hepatic Injury in a Subject

Another aspect of the invention pertains to methods for treating or preventing hepatic injury in a subject in need thereof. The method includes administering to the subject a P-glycoprotein inhibitor in an amount effective to treat or prevent hepatic injury in the subject. The method can also involve selecting a subject in need of treatment for or prevention of hepatic injury, prior to the administration of the P-glycoprotein inhibitor to the subject.

A hepatic injury can be any injury to the liver, such as an injury to the liver that interferes with the normal function of the liver. The hepatic injury may involve an injury due to the over- or under-production of hepatic enzymes, e.g, alanine aminotransferase, aspartate aminotransferase, or γ-glutammyl transferase, in the liver. For example, the hepatic injury may be hepatic fibrosis, hepatic cirrhosis, hepatic injury caused by a drug, hepatic injury due to prolonged ethanol uptake, or hepatic injury due to carbon tetrachloride exposure.

The methods of the invention include administering, e.g., dispensing, delivering or applying, to a subject a P-glycoprotein inhibitor, e.g., a P-glycoprotein inhibitor in a pharmaceutical formulation, or a cytochrome P450 inhibitor by any suitable route for delivery of the composition to the desired location in the subject, including delivery by either the parenteral or oral route, intramuscular injection, subcutaneous/intradermal injection, intravenous injection, buccal administration, transdermal delivery and administration by the rectal, colonic, intranasal or respiratory tract route. The P-glycoprotein inhibitor and the cytochrome P450 inhibitor can be administered in the same formulation or in separate formulations. In other preferred embodiments, the P-glycoprotein inhibitor and the cytochrome P450 inhibitor are administered simultaneously. In yet other preferred embodiments, the P-glycoprotein inhibitor and the cytochrome P450 inhibitor are administered at different times.

As used herein, the term “effective amount” includes an amount effective, at dosages and for periods of time necessary, to achieve the desired result, e.g., sufficient to treat or prevent hepatic injury in a subject. An effective amount of a P-glycoprotein inhibitor, as defined herein may vary according to factors such as the disease state, age, and weight of the subject, and the ability of the P-glycoprotein inhibitor to elicit a desired response in the subject. Dosage regimens may be adjusted to provide the optimum therapeutic response. An effective amount is also one in which any toxic or detrimental effects of the P-glycoprotein inhibitor are outweighed by the therapeutically beneficial effects. For example, the P-glycoprotein inhibitor is administered to the subject in an amount of about 10-100 mg/kg, about 10-60 mg/kg, or 10-40 mg/kg. The P-glycoprotein inhibitor may be administered to the subject in an amount of 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, or 65 mg/kg. Ranges intermediate to the above recited values, e.g., about 20-40 mg/kg or about 40-60 mg/kg, also are intended to be part of this invention. For example, ranges of span values using a combination of any of the above recited values as upper and/or lower limits are intended to be included.

In one embodiment, the method of the invention includes administering to a subject a P-glycoprotein inhibitor in combination with a cytochrome P450 inhibitor, e.g, a member of the cytochrome P450 family, e.g., CPY1, CYP2, and CYP3, which is involved in drug metabolism. Cytochrome P450 family members can be found in the liver as well as in the enterocytes lining the lumen of the intestine. Several of the cytochrome P450 family members are inducible, i.e., their concentration as well as their catalytic activity is increased after exposure of an individual to particular classes of drugs, endogenous compounds, and environmental agents. Cytochrome P450 family members are described in, for example, Watkins P. B. et al. (1992) Gastroenterology Clinics of North America 21(3):511-526, the contents of which are incorporated herein by reference.

Cytochrome P450 inhibitors are known in the art and include calcium channel blockers, e.g., Verapamil, Felodipine, or Diltiazem; flavanoids, e.g., Quercetin, Kaempherol, or Benzoflavone; steroid hormones, e.g., cortisol, or progesterone; chemotherapeutic agents; or antidiabetic agents, e.g., Tolbutamide. Cytochrome P450 inhibitors are described in, for example, PCT Application No. WO 95/20980, the contents of which are incorporated herein by reference.

In a further embodiment, the hepatic injury is caused by a drug and the P-glycoprotein inhibitor is administered to the subject simultaneously with the drug, within 1, 2, 3, 4, 5, 6, 7, 8, 9, 10, 11, or 12 hours after the drug is administered to the subject, or within 1, 2, 3, 4, 5, 6, 7, 8, 9, 10, 11, or 12 hours before the drug is administered to the subject. Ranges of span values using a combination of any of the above recited values as upper and/or lower limits are intended to be included, e.g., 1-6 hours or 4-10 hours.

As used herein, the term “drug” is intended to encompass all types of pharmaceutical compounds and includes antibiotics, enzymes, chemical compounds, e.g., carbon tetrachloride, mixtures of chemical compounds, biological macromolecules, e.g., peptides, and analogs thereof. Similar substances are known or can be readily ascertained by one of skill in the art. Drugs intended to be encompassed include those described in Harrison's Principles of Internal Medicine, Thirteenth Edition, Eds. T. R. Harrison et al. McGraw-Hill N.Y., N.Y.; and the Physicians Desk Reference 50th Edition 1997, Oradell N.J., Medical Economics Co., the complete contents of which are expressly incorporated herein by reference.

In one embodiment, the drug is a hydrophobic peptide such as a β-amyloid peptide derivative, e.g., PPI-558, PPI-657, PPI-1019, PPI-578, or PPI-655. As used herein, the term “β-amyloid peptide derivative” includes peptides derived from the natural β-amyloid peptide (β-AP). Natural β-AP is derived by proteolysis of a larger protein called the amyloid precursor protein (APP) described in Kang, J. et al. (1987) Nature 325:733; Goldgaber, D. et al. (1987) Science 235:877; Robakis, N. K. et al. (1987) Proc. Natl. Acad. Sci. USA 84:4190; Tanzi, R. E. et al. (1987) Science 235:880. Differential splicing of the APP messenger RNA leads to at least five forms of APP, composed of either 563 amino acids (APP-563), 695 amino acids (APP-695), 714 amino acids (APP-714), 751 amino acids (APP-751) or 770 amino acids (APP-770). Within APP, naturally-occurring β amyloid peptide begins at an aspartic acid residue at amino acid position 672 of APP-770. Naturally-occurring β-AP derived from proteolysis of APP is 39 to 43 amino acid residues in length, depending on the carboxy-terminal end point, which exhibits heterogeneity. The predominant circulating form of β-AP in the blood and cerebrospinal fluid of both AD patients and normal adults is β1-40 (“short β”). Seubert, P. et al. (1992) Nature 359:325; Shoji, M. et al. (1992) Science 258:126. However, P1-42 and β1-43 (“long β”) also are forms in β-amyloid plaques. Masters, C. et al. (1985) Proc. Natl. Acad. Sci. USA 82:4245; Miller, D. et al. (1993) Arch. Biochem. Biophys. 301:41; Mori, H. et al. (1992) J. Biol. Chem. 267:17082. β-amyloid peptide derivatives are described in detail in subsection II below and also described in PCT Application Nos. WO 96/28471 and WO 98/08868, the contents of which are incorporated herein by reference.

In another aspect, the invention features a method for modulating, e.g., decreasing, the levels of a hepatic enzyme in a subject. The method includes administering to the subject a P-glycoprotein inhibitor in an amount effective to modulate the levels of a hepatic enzyme in the subject. The method can also involve selecting a subject in need of modulation of hepatic enzymes, prior to the administration of the P-glycoprotein inhibitor to the subject.

As used herein, the term “hepatic enzyme” includes an enzyme that is secreted and/or functions in the liver. For example, the hepatic enzyme can be alanine aminotransferase, aspartate aminotransferase, or γ-glutammyl transferase.

VII. Pharmaceutical Compositions

Another aspect of the invention pertains to pharmaceutical compositions of the hydrophobic peptides, e.g., the β-amyloid peptide derivatives, of the invention. In one embodiment, the composition includes a hydrophobic peptide, e.g., a β-amyloid peptide derivative, in an amount sufficient to, for example, inhibit P-glycoprotein and/ or cytochrome P450 function, and allow a drug to cross the blood brain barrier and enter the brain, and a pharmaceutically acceptable carrier. In another embodiment, the composition includes a hydrophobic peptide, e.g., a β-amyloid peptide derivative, in an amount sufficient to, for example, inhibit P-glycoprotein and/or cytochrome P450 function, and to allow a drug to be transported across the gastrointestinal tract and enter the bloodstream, and a pharmaceutically acceptable carrier.

Another aspect of the invention pertains to pharmaceutical compositions which include a P-glycoprotein inhibitor and a drug, wherein the drug is present in an amount effective to treat a targeted condition in a subject and the P-glycoprotein inhibitor is present in an amount effective to prevent hepatic injury in the subject. In one embodiment, the pharmaceutical composition further includes a cytochrome P450 inhibitor. In another embodiment, the pharmaceutical composition further includes a pharmaceutically acceptable carrier, e.g., a lipid-based carrier.

An “effective amount” refers to an amount effective, at dosages and for periods of time necessary, to achieve a desired physiological result, e.g., inhibition of P-glycoprotein and/or cytochrome P450 function or prevention of hepatic injury in a subject. A therapeutically effective amount may vary according to factors such as the disease state, age, sex, and weight of the individual, and the ability of the hydrophobic peptide, e.g., the β-amyloid peptide derivative, or the P-glycoprotein inhibitor to elicit a desired response in the individual. Dosage regimens may be adjusted to provide the optimum physiological response. A therapeutically effective amount is also one in which any toxic or detrimental effects of the hydrophobic peptide, e.g., a β-amyloid peptide derivative, or the P-glycoprotein inhibitor are outweighed by the therapeutically beneficial effects. The potential neurotoxicity of the hydrophobic peptides, e.g., the β-amyloid peptide derivatives, or the P-glycoprotein inhibitors of the invention can be assayed using the art known cell-based assays and a therapeutically effective hydrophobic peptide or P-glycoprotein inhibitor can be selected which does not exhibit significant neurotoxicity. In a preferred embodiment, a therapeutically effective amount of a hydrophobic peptide, e.g., a β-amyloid peptide derivative, or P-glycoprotein inhibitor is sufficient to alter, and preferably inhibit, P-glycoprotein and/or cytochrome P450 function.

A non-limiting range for an effective amount of a hydrophobic peptide, e.g., a β-amyloid peptide derivative, or a P-glycoprotein inhibitor is 100 nM-20 μM. It is to be noted that dosage values may vary with the severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The amount of hydrophobic peptide, e.g., β-amyloid peptide derivative, or P-glycoprotein inhibitor in the composition may vary according to factors such as the disease state, age, sex, and weight of the individual, each of which may affect the amount of P-glycoprotein and/or cytochrome P450 in the individual. Dosage regimens may be adjusted to provide the optimum physiological response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of hydrophobic peptide, e.g., β-amyloid peptide derivative, or P-glycoprotein inhibitor calculated to produce the desired effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

As used herein “pharmaceutically acceptable carrier” includes any and all solvents, dispersion media, coatings, antibacterial and antifungal agents, isotonic and absorption delaying agents, and the like that are physiologically compatible. In one embodiment, the carrier is suitable for parenteral administration. Preferably, the carrier is suitable for administration into the central nervous system (e.g., intraspinally or intracerebrally). Alternatively, the carrier can be suitable for intravenous, intraperitoneal or intramuscular administration. In another embodiment, the carrier is suitable for oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as manitol, sorbitol, or sodium chloride in the composition. Prolonged absorption of the injectable compositions can be brought about by including in the composition an agent which delays absorption, for example, monostearate salts and gelatin. Moreover, the peptides, e.g., the β-amyloid peptide derivatives, can be administered in a time release formulation, for example in a composition which includes a slow release polymer. The active peptide or P-glycoprotein inhibitor can be prepared with carriers that will protect the peptide or P-glycoprotein inhibitor against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG). Many methods for the preparation of such formulations are patented or generally known to those skilled in the art.

Sterile injectable solutions can be prepared by incorporating the active peptide or P-glycoprotein inhibitor in the required amount in an appropriate solvent with one or a combination of ingredients enumerated above, as required, followed by filtered sterilization. Generally, dispersions are prepared by incorporating the peptide or P-glycoprotein inhibitor into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

A hydrophobic peptide or P-glycoprotein inhibitor can be formulated with one or more additional compounds that enhance the solubility of the hydrophobic peptide or P-glycoprotein inhibitor. Preferred compounds to be added to formulations to enhance the solubility of the hydrophobic peptides, e.g, the β-amyloid peptide derivatives, are cyclodextrin derivatives, preferably hydroxypropyl-γ-cyclodextrin. Drug delivery vehicles containing a cyclodextrin derivative for delivery of peptides to the central nervous system are described in Bodor, N., et al. (1992) Science 257:1698-1700. For the the hydrophobic peptides, e.g., the β-amyloid peptide derivatives, described herein, inclusion in the formulation of hydroxypropyl-γ-cyclodextrin at a concentration 50-200 mM increases the aqueous solubility of the hydrophobic peptides, e.g., the β-amyloid peptide derivatives. In addition to increased solubility, inclusion of a cyclodextrin derivative in the formulation may have other beneficial effects, since β-cyclodextrin itself has been reported to interact with the Aβ peptide and inhibit fibril formation in vitro (Camilleri, P., et al. (1994) FEBS Letters 341:256-258. Accordingly, use of a hydrophobic peptide, e.g, a β-amyloid peptide derivative, of the invention in combination with a cyclodextrin derivative may result in greater inhibition of Aβ aggregation than use of the hydrophobic peptide, e.g., the β-amyloid peptide derivative alone. Chemical modifications of cyclodextrins are known in the art (Hanessian, S., et al. (1995) J. Org Chem. 60:4786-4797). In addition to use as an additive in a pharmaceutical composition containing a β-amyloid peptide derivative of the invention, cyclodextrin derivatives may also be useful as modifying groups and, accordingly, may also be covalently coupled to an Aβ peptide compound to form a hydrophobic peptide, e.g., a β-amyloid peptide derivative, of the invention.

Another preferred formulation for the peptide or P-glycoprotein inhibitor comprises the detergent Tween-80, polyethylene glycol (PEG) and ethanol in a saline solution. A non-limiting example of such a preferred formulation is 0.16% Tween-80, 1.3% PEG-3000 and 2% ethanol in saline.

A hydrophobic peptide or P-glycoprotein inhibitor can be formulated into a pharmaceutical composition wherein the hydrophobic peptide or the p-glycoprotein inhibitor is the only active compound or, alternatively, the pharmaceutical composition can contain additional active compounds. For example, a hydrophobic peptide or P-glycoprotein inhibitor can be formulated in combination with other P-glycoprotein and/or cytochrome P450 inhibitors.

The hydrophobic peptides or P-glycoprotein inhibitors can further be formulated in combination with a particular drug of interest. The drug can be an agent suitable for treating a targeted condition in a subject, e.g., a targeted condition of the brain, and capable of being delivered in active form, in vivo using the methods of the invention. The ordinarily skilled artisan would be able to select appropriate art-recognized drugs for a particular disease or condition targeted for treatment. Examples of such drugs include antibiotics, enzymes, chemical compounds, mixtures of chemical compounds, biological macromolecules, e.g., peptides, and analogs thereof. Similar substances are known or can be readily ascertained by one of skill in the art. One skilled in the art can look to Harrison's Principles of Internal Medicine, Thirteenth Edition, Eds. T. R. Harrison et al. McGraw-Hill N.Y., N.Y.; and the Physicians Desk Reference 50th Edition 1997, Oradell N.J., Medical Economics Co., the complete contents of which are expressly incorporated herein by reference, to determine appropriate drugs for administration to a subject.

Moreover, two or more hydrophobic peptides, e.g., β-amyloid peptide derivatives, or two or more P-glycoprotein inhibitors may be used in combination. Moreover, a hydrophobic peptide, e.g., a β-amyloid peptide derivative, of the invention can be combined with one or more other agents that have anti-amyloidogenic properties. For example, a hydrophobic peptide, e.g., a β-amyloid peptide derivative, can be combined with the non-specific cholinesterase inhibitor tacrine (COGNEX®, Parke-Davis) or aricett.

In another embodiment, a pharmaceutical composition of the invention is provided as a packaged formulation. The packaged formulation may include a pharmaceutical composition of the invention in a container and printed instructions for administration of the composition for treating a subject having a CNS disorder, e.g Alzheimer's disease, or a hepatic injury.

VIII. Screening Assays for P-glycoprotein and/or Cytochrome P450 Binding and Inhibition

The invention also features a method for identifying a hydrophobic peptide, e.g., a β-amyloid peptide derivative, useful for increasing bioavailability, e.g., bioavailability in the brain or oral bioavailability, of a drug in a subject. The method includes screening a candidate hydrophobic peptide for the ability to bind to P-glycoprotein and/or cytochrome P450 and inhibit its function, and selecting a hydrophobic peptide which binds to P-glycoprotein and/or cytochrome P450 and inhibits its function, thereby identifying a hydrophobic peptide, e.g., a β-amyloid peptide derivative, useful for increasing bioavailability of a drug in a subject.

For example, the ability of the candidate hydrophobic peptide to bind the P-glycoprotein and/or cytochrome P450 can be accomplished by, for example, coupling the hydrophobic peptide with a radioisotope or enzymatic label such that binding of the hydrophobic peptide to the P-glycoprotein and/or cytochrome P450 can be determined by detecting the labeled hydrophobic peptide in a complex. For example, hydrophobic peptides can be labeled with ¹²⁵I, ³⁵S, ¹⁴C, or ³H, either directly or indirectly, and the radioisotope detected by direct counting of radioemmission or by scintillation counting. Alternatively, hydrophobic peptides can be enzymatically labeled with, for example, horseradish peroxidase, alkaline phosphatase, or luciferase, and the enzymatic label detected by determination of conversion of an appropriate substrate to product.

Moreover, the ability of the test hydrophobic peptide to inhibit P-glycoprotein activity can be determined, for example, by using drug transport assays as described in U.S. Pat. No. 5,567,592, the contents of which are incorporated herein by reference. Briefly, drug transport assays include measuring the transport of drugs into brush border membrane vesicles (prepared as described in Hsing S. et al. (1992) Gastroenterology 102:879-885) in an ATP-dependent fashion. Uptake of the drug in the presence of ATP is monitored using fluorescence or absorbance techniques, for instance using Rh 123 as the fluorescence drug transported into the interior of the vesicle. Radioactively labeled drugs can also be used to monitor drug transport into the interior of the vesicle using a filter wash system. The addition of ATP induces the transport of the drug into the vesicle and increases drug transport compared to passive diffusion of the drug into the vesicle interior. Addition of non-hydrolyzable analogs of ATP such as ATP gamma S or adenosine monophosphate para-nitrophenol (AMP-PNP) will not produce an ATP dependent influx of drug into the vesicle. Thus, the introduction of a non-hydrolyzable nucleotide actually occures due to ATP hydrolysis from the P-glycoprotein transport system.

The addition of a candidate hydrophobic peptide (a candidate β-amyloid peptide derivative) to this assay system using a fluorescent drug or radioactive drug and monitoring its uptake, will reduce the uptake of the drug into the interior of the vesicle with the addition of ATP. This reduction in drug transport respresents an increase of the bioavailability of the drug. The vesicles transporting drugs in an ATP dependent fashion are oriented with the cystolic face of the P-glycoprotein accessible to the ATP. It is these vesicles that hydrolyze the ATP and transport the drug into the interior of the vesicle. The interior of the vesicle in turn corresponds to the luminal surface or the apical membrane of the brush border cells. Thus, transport into the lumen of the vesicle or interior of the vesicle corresponds to transport into the lumen to the gut. A decrease in the transport of the lumen of the vesicle is the equivalent of increasing net drug absorption and increasing the drug bioavailability.

In another embodiment, the ability of the test hydrophobic peptide to inhibit P-glycoprotein activity can be determined using cultured brain capillary endothelial cells, as described in, for example, Tatsuta T. et al. (1992) J. Biol. Chem. 267:20383-91 and Biegel D. et al. (1995) Brain Res. 692:183-9, the contents of which are incorporated herein by reference.

In yet another embodiment, the ability of the test hydrophobic peptide to inhibit P-glycoprotein activity can be determined in vivo using the P-glycoprotein knockout mice developed by Schinkel A. H. et al. (1994) Cell 77:491-502, the contents of which are incorporated herein by reference.

The ability of the test hydrophobic peptide to inhibit cytochrome P450 function can be determined by, for example, using cultured cells of either hepatocytes or enterocytes, or freshly prepared cells from either the liver or the gut. Various methods of gut epithelial cell isolation can be used, such as the method of Watkins et al. (1985) J. Clin. Invest. 80:1029-36. The production of cytochrome P450 metabolites in these cells can be measured using high pressure liquid chromatography (HPLC). Cytochrome P450 activity can also be assayed by calorimetrically measuring erythromycin demethylase activity as described in Wrighton et al. (1985) Mol. Pharmacol. 28:312-321.

IX. Kits

In another aspect, the invention features a kit which includes a hydrophobic peptide, e.g., a β-amyloid peptide derivative, and instructions for use in increasing the bioavailability of a drug. In another embodiment, the kit can include a hydrophobic peptide, e.g., a β-amyloid peptide derivative, a drug, and instructions for use in increasing the bioavailability of a drug. In yet another embodiment, the kit can include a hydrophobic peptide, e.g., a β-amyloid peptide derivative, a P-glycoprotein inhibitor, and instructions for use in increasing the bioavailability of a drug. In yet a further embodiment, the kit can include a hydrophobic peptide, e.g., a β-amyloid peptide derivative, a drug, a cytochrome P450 inhibitor, and instructions for use in increasing the bioavailability of a drug.

In another aspect, the invention features a kit including a P-glycoprotein inhibitor, a drug, and instructions for administration to a subject in an amount effective to treat a targeted condition in the subject and prevent a hepatotoxic effect of the drug to the subject. In one embodiment, the kit further includes a cytochrome P450 inhibitor.

The following examples which further illustrate the invention should not be construed as limiting. The contents of all references, patents and published patent applications cited throughout this application, as well as the Figures are incorporated herein by reference.

### EXAMPLES

Example 1

Preparation of β-amyloidpeptide Derivatives

β-amyloid peptide derivatives comprising D-amino acids can be prepared by solid-phase peptide synthesis, for example using an N^{α}-9-fluorenylmethyloxycarbonyl (FMOC)-based protection strategy as follows. Starting with 2.5 mmoles of FMOC-D-Val-Wang resin, sequential additions of each amino acid are performed using a four-fold excess of protected amino acids, 1-hydroxybenzotriazole (HOBt) and diisopropyl carbodiimide (DIC). Recouplings are performed when necessary as determined by ninhydrin testing of the resin after coupling. Each synthesis cycle is minimally described by a three minute deprotection (25% piperidine/N-methyl-pyrrolidone (NMP)), a 15 minute deprotection, five one minute NMP washes, a 60 minute coupling cycle, five NMP washes and a ninhydrin test. For N-terminal modification, an N-terminal modifying reagent is substituted for an FMOC-D-amino acid and coupled to a 700 mg portion of the fully assembled peptide-resin by the above protocol. The peptide is removed from the resin by treatment with trifluoroacetic acid (TFA) (82.5%), water (5%), thioanisole (5%), phenol (5%), ethanedithiol (2.5%) for two hours followed by precipitation of the peptide in cold ether. The solid is pelleted by centrifugation (2400 rpm×10 min.), and the ether decanted. The solid is resuspended in ether, pelleted and decanted a second time. The solid is dissolved in 10% acetic acid and lyophilized to dryness. For purification and analysis, 60 mg of the solid is dissolved in 25% acetonitrile (ACN)/0.1% TFA and applied to a C18 reversed phase high performance liquid chromatography (HPLC) column.

Alternatively, β-amyloid peptide derivatives comprising D-amino acids can be prepared on an Advanced ChemTech Model 396 multiple peptide synthesizer using an automated protocol established by the manufacturer for 0.025 mmole scale synthesis. Double couplings are performed on all cycles using 2-(1H-benzotriazol-1-yl)-1,1,3,3-tetramethyluronium hexafluorophosphate (HBTU)/N,N-diisopropylethylamine (DIEA)/HOBt/FMOC-D-amino acid in four-fold excess for 30 minutes followed by DIC/HOBt/FMOC-D-amino acid in four-fold excess for 45 minutes. The peptide is deprotected and removed from the resin by treatment with TFA/water (95%/5%) for three hours and precipitated with ether as described above. The pellet is resuspended in 10% acetic acid and lyophilized. The material is purified by a preparative HPLC using 15%-40% acetonitrile over 80 minutes on a Vydac C18 column (21×250 mm).

Various N-terminally modified β-amyloid peptide derivatives can be synthesized using standard methods. Fully-protected resin-bound peptides are prepared as described above on Wang resin to eventually afford carboxyl terminal peptide acids. Small portions of each peptide resin (e.g., 13-20 μmoles) are aliquoted into the wells of the reaction block of an Advanced ChemTech Model 396 Multiple Peptide Synthesizer. The N-terminal FMOC protecting group of each sample is removed in the standard manner with 25% piperidine in NMP followed by extensive washing with NMP. The unprotected N-terminal α-amino group of each peptide-resin sample can be modified using one of the following methods:

Method A, coupling of modifying reagents containing free carboxylic acid groups: The modifying reagent (five equivalents) is predissolved in NMP, DMSO or a mixture of these two solvents. HOBT and DIC (five equivalents of each reagent) are added to the dissolved modifier and the resulting solution is added to one equivalent of free-amino peptide-resin. Coupling is allowed to proceed overnight, followed by washing. If a ninhydrin test on a small sample of peptide-resin shows that coupling is not complete, the coupling is repeated using 1-hydroxy-7-azabenzotriazole (HOAt) in place of HOBt.

Method B, coupling of modifying reagents obtained in preactivated forms: The modifying reagent (five equivalents) is predissolved in NMP, DMSO or a mixture of these two solvents and added to one equivalent of peptide-resin. Diisopropylethylamine (DIEA; six equivalents) is added to the suspension of activated modifier and peptide-resin. Coupling is allowed to proceed overnight, followed by washing. If a ninhydrin test on a small sample of peptide-resin shows that coupling is not complete, the coupling is repeated.

After the second coupling (if required) the N-terminally modified peptide-resins are dried at reduced pressure and cleaved from the resin with removal of side-chain protecting groups as described above. Analytical reversed-phase HPLC is used to confirm that a major product is present in the resulting crude peptides, which are purified using Millipore Sep-Pak cartridges or preparative reverse-phase HPLC. Mass spectrometry or high-field nuclear magnetic resonance spectrometry is used to confirm the presence of the desired compound in the product.

### Example 2

Biodistribution of β-amyloid Peptide Derivatives

Methods

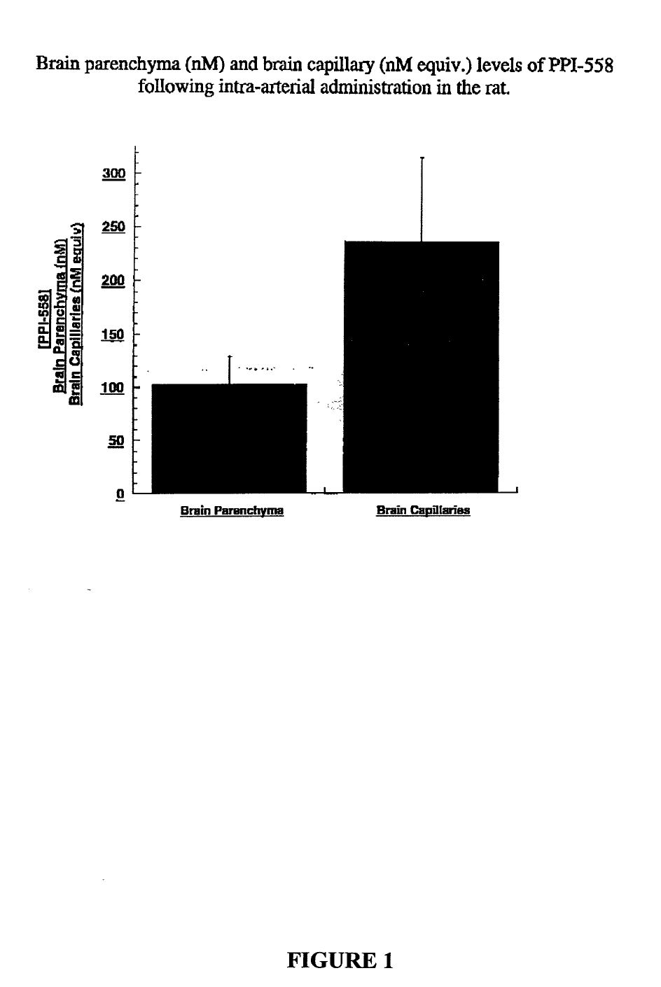

In experiment 1, the results of which are depicted in FIG. 1, male Sprague-Dawley rats under Ketamine/xylazine anesthesia, received a 10 minute intra-arterial infusion of ³H-PPI-588/¹⁴C-sucrose. The drug cocktail was infused (100 μL/h) via a cannula in the external carotid artery so that the infusate was picked up by the blood and carried directly to the Circle of Willis/brain (via the left internal carotid artery, PTA occluded). At 30 minutes post cessation of the 10 minute IA infusion the left side of the brain was voided of blood by manually infusing I ml of saline via the cannula in the external carotid artery with the common carotid artery now ligated. The perfused left forebrain (choroid plexus removed) was subjected to capillary depiction (as described by Triguero et al., 1990) so as to produce brain supernatant that was void of blood vessels (as determined by alkaline phosphatase measurements). The concentration of parent PPI-558 (nM) that has crossed the blood brain barrier was determined by can extraction/HPLC analysis.

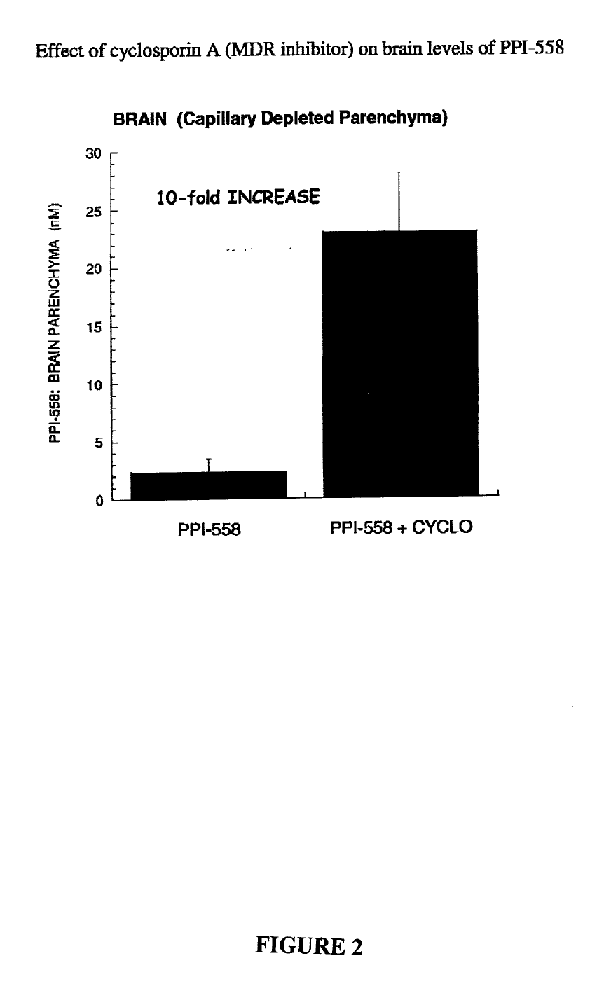

In experiment 2, the results of which are depicted in FIG. 2, male Sprague-Dawley rats under ketamine/xylazine anesthesia, received an intravenous bolus of cyclasporin A (50 mg/kg) 30 min. prior to receiving a 1 minute intravenous infusion of ³H-PPI-558 (approx. 3 mg/kg). At 1 hour following PPPI-558 administration the left side of the brain was voided of blood by manually infusing 1 mL of saline via a cannula in the common carotid artery, with the external carotid artery ligated. The perfused, ipsilateral, left forebrain (choroid plexus removed) was subjected to capillary depletion (as described by Triguero et al., 1990) so as to produce brain supernatant that was void of blood vessels (as determined by alkaline phosphatase measurements). The concentration of parent PPI-558 (nM) that had crossed the blood brain barrier was determined by LC/MS/MS and/or CAN extraction/HPLC analysis.

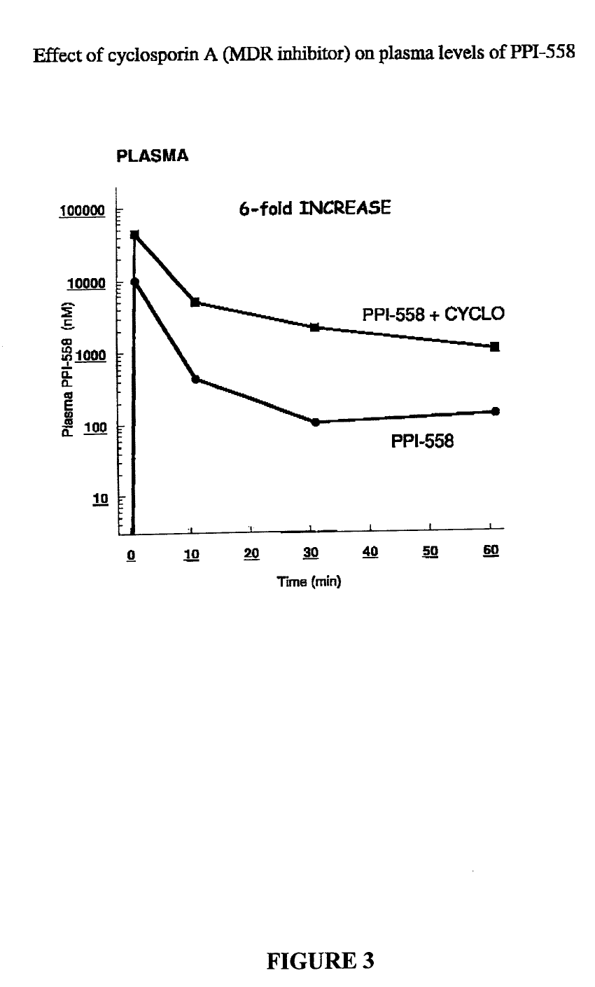

In experiment 3, the results of which are depicted in FIG. 3, plasma samples were obtained from the animals in FIG. 2 at 1, 20, 30 and 60 minutes post administration of ³H-PPI-558. Parent levels of PPI-558 determined by ACN extraction/HPLC analysis.

In experiment 4, the results of which are depicted in Table III, the levels of ³H-PPI-558 were determined in various organs/tissues in the animals shown in FIG. 2. The amount of radioactivity was determined from representative samples of organs (approximately 100 mg) by scintillation counting.

Results

The first indication that P-glycoproteins are involved in the brain uptake of the β-amyloid peptide derivative PPI-558, came from the observation that brain levels of >100 nM were achievable when administered directly to the brain, via the internal carotid artery (see FIG. 1) but were very low (<5 nM) when PPI-558 was administered intravenously (IV), subcutaneously (SC) or intramuscularly (IM). Upon review of the data, it was observed that very high levels of PPI-558 was capillary bound (approx. 250 nM equiv.) following IA administration versus very low levels following IV, SC or IM. This indicated that PPI-558 may be saturating the efflux system (P-glycoproteins) and, thus, allowing PPI-558 to remain in the brain parenchyma.

The effect of inhibiting the P-glycoprotein efflux pump in the blood brain barrier by using cyclosporin A (a P-glycoprotein inhibitor), was then investigated in experiments using the β-amyloid peptide derivatives PPI-558 and PPI-1019. The general experimental procedure was that used by Hendrikse et al. (1998) Br. J. Pharmacol. 124:1413-1418.

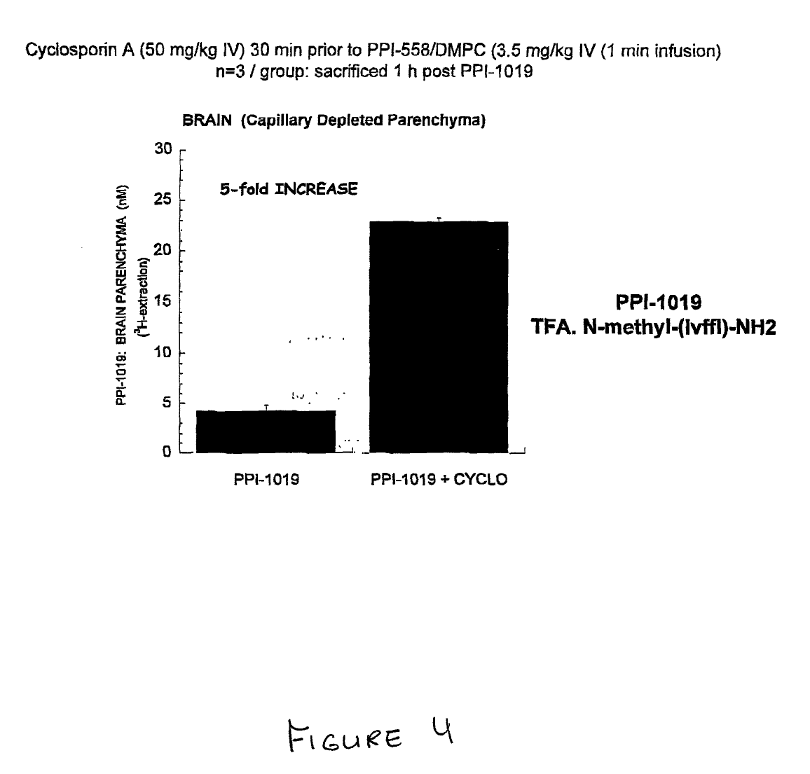

The data shown in FIG. 2 demonstrates that brain levels of PPI-558 were elevated 10-fold in the presence of cyclosporin A. The data shown in FIG. 4 demonstrates that brain levels of PPI-1019 were elevated 5-fold in the presence of cyclosporin A. Table II demonstrates the results from an analysis of various β-amyloid peptide derivatives for the ability to be transported in the brain in the presence of a P-glycoprotein inhibitor, and the ability to inhibit β-amyloid aggregation.

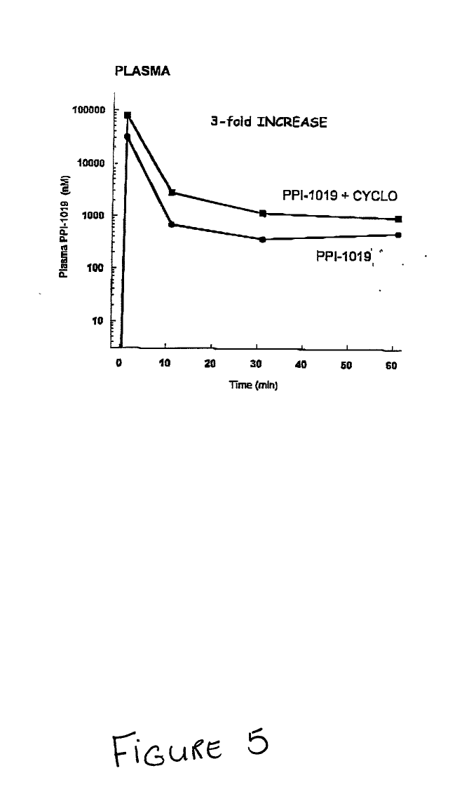

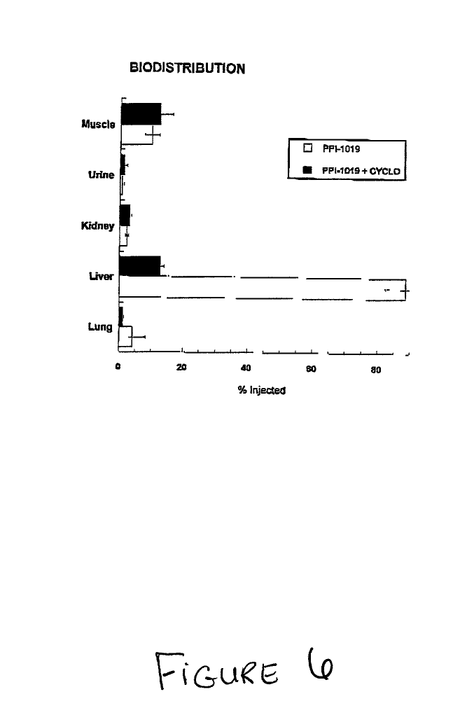

Plasma levels (FIGS. 3 and 5) were also elevated and may contribute to the higher brain levels observed. The biodistribution data with PPI-558 (Table III) demonstrate higher levels were observed within the small intestine in the presence of cyclosporin A. This indicates an increase in bioavailability if the β-amyloid peptide derivatives of the invention plus other pharmacological agents that have affinity for P-glycoproteins are co-administered orally. Furthermore, the observation of decreased levels of PPI-1019 within the liver in the presence of cyclosporin A (FIG. 6) demonstrates the ability of the P-glycoprotein inhibitor to down-modulate liver accumulation of a coadministered drug, and as a consequence down-modulate liver toxicity due to the drug. It was also observed that hepatic enzyme levels were decreased when the β-amyloid peptide derivative was co-administered with a P-glycoprotein inhibitor (e.g., cyclosporin A).

TABLE II
Potent B-amyloid Aggregation Inhibitors

| Com- | | K_{D} | | Solubility | Tested with |
|---|---|---|---|---|---|
| pound | Structure | (nM) | MW | (mg/ml) | MDR Inhibitor |
| PPI-558 | 4-HBz-lvffa-NH₂ | 2.82 | 715 | .001 | + |
| PPI-465 | H-lvffa-NH₂ | 0.92 | 595 | 1.49 | |
| PPI-657 | H-lvffv-NH₂ | 0.33 | 623 | 0.97 | + |
| PPI-578 | H-lffvl-NH₂ | 0.29 | 637 | 2.5 | |
| PPI-1007 | (CH3)2-lvffl-NH₂ | 0.86 | 665 | 0.18 | |
| PPI-1019 | CH3-lvffl-NH₂ | 0.50 | 765 | 1.0 | + |
| PPI-1125 | (H-lvf-NH-)2 | 1.27 | 751 | 5 | |

TABLE III
Biodistribution of 3H-PPI-558 +/− Cyclosporin A
BIODISTRIBUTION: EFFECT OF MDR INHIBITORS ON PPI-558
1 mm (1 mL) IV Infusion: sac @ t = 61 min

| | Organ/Tissue/Fluid (expressed as % administered) | | | | | | | | | | | | | | | | | |
|---|---|---|---|---|---|---|---|---|---|---|---|---|---|---|---|---|---|---|
| | | | | Kid- | Duo- | Mus- | | | Jug- | | | | Pan- | Adre- | blood | | Car- | SUM |
| BUI # | Heart | Lung | Liver | ney | denum | cle | Spleen | Fat | ular | Ileum | Colon | Tongue | creas | nals | at sac | Urine | cass | % |

PPI-558/DMPC (1:30) approx. 3 mg/kg IV

| | | | | | | | | | | | | | | | | | | |
|---|---|---|---|---|---|---|---|---|---|---|---|---|---|---|---|---|---|---|
| 66 | 0.0 | 0.5 | 62.3 | 0.7 | 0.2 | 2.5 | 0.5 | 0.5 | 0.0 | 0.9 | 0.0 | 0.0 | 0.0 | 0.0 | 0.3 | 10 | 4 | 73 |
| 67 | 0.1 | 0.3 | 58.7 | 1.7 | 0.2 | 4.1 | 0.3 | 0.6 | 0.0 | 1.0 | 0.1 | 0.0 | 0.0 | 0.0 | 0.8 | 12 | 6 | 75 |
| 68 | | 0.2 | 58.4 | 0.5 | | 3.0 | | | | | | | | | | | | |
| MEAN | 0.07 | 0.33 | 59.82 | 0.95 | 0.16 | 3.18 | 0.43 | 0.54 | 0.02 | 0.95 | 0.06 | 0.02 | 0.03 | 0.00 | 0.52 | 1.09 | 5.00 | 74.23 |

CYCLOSPORIN A (50 mg/kg IV t = −30 min) PPI-558/DMPC (1:30)

| | | | | | | | | | | | | | | | | | | |
|---|---|---|---|---|---|---|---|---|---|---|---|---|---|---|---|---|---|---|
| 67 | 0.2 | 0.5 | 25.8 | 2.5 | 0.9 | 16.7 | 0.4 | 1.3 | 0.0 | 1.7 | 0.2 | 0.1 | 0.2 | 0.0 | 3.0 | 1.5 | 21 | 76 |
| 68 | | 0.4 | 22.8 | 2.7 | | 17.8 | | | | | | | | | | | | |
| MEAN | 0.24 | 0.44 | 24.31 | 2.59 | 0.89 | 17.25 | 0.36 | 1.32 | 0.00 | 1.65 | 0.23 | 0.08 | 0.20 | 0.02 | 3.03 | 1.54 | 21.17 | 76.21 |
| FOLD IN- | 3 | 1 | 0.4 | 3 | 6 | 5 | 1 | 2 | | 2 | 4 | 5 | 7 | | 6 | 1 | 4 | |
| CREASE | | | | | | | | | | | | | | | | | | |

### Equivalents

Those skilled in the art will recognize, or be able to ascertain, using no more than routine experimentation, many equivalents to the specific embodiments of the invention described herein. Such equivalents are intended to be encompassed by the following claims.

1. A method for enhancing the bioavailability of a drug in a subject, comprising administering to the subject a hydrophobic peptide in an amount sufficient to enhance the bioavailability of the drug in the subject.

2. The method of claim 1, wherein the hydrophobic peptide is a β-amyloid peptide derivative.

3. The method of claim 2, wherein the β-amyloid peptide derivative is selected from the group consisting of PPI-558, PPI-657, PPI-1019, PPI-578, and PPI-655.

4. The method of claim 3, wherein the β-amyloid peptide derivative is PPI-1019.

5. The method of claim 1, wherein the drug and the hydrophobic peptide are administered to the subject simultaneously.

6. The method of claim 1, wherein the drug and the hydrophobic peptide are administered to the subject at different times.

7. The method of claim 1, further comprising administering to the subject a P-glycoprotein inhibitor.

8. The method of claim 7, wherein the P-glycoprotein inhibitor is selected from the group consisting of antiarrhythmics, antibiotics, antifungals, calcium channel blockers, cancer chemotherapeutics, hormones, antiparasites, local anesthetics, phenothiazines, and tricyclic antidepressants.

9. The method of claim 1, further comprising administering to the subject a cytochrome P450 inhibitor.

10. The method of claim 1, wherein the bioavailability of the drug is enhanced in the brain of the subject.

11. The method of claim 10, wherein the subject is suffering from a CNS disorder.

12. The method of claim 11, wherein the CNS disorder is a neurodegenerative disorder.

13. The method of claim 11, wherein the CNS disorder is Alzheimer's disease.

14. The method of claim 1, wherein the drug inhibits aggregation of natural β-amyloid peptide.

15. The method of claim 1, wherein the oral bioavailability of the drug is enhanced in the subject.

16. The method of claim 1, wherein the β-amyloid peptide derivative is administered to the subject intravenously.

17. The method of claim 1, wherein the β-amyloid peptide derivative is administered to the subject intramuscularly.

18. The method of claim 1, wherein the β-amyloid peptide derivative is administered to the subject subcutaneously.

19. The method of claim 1, wherein the subject is a human.

20. A method for enhancing the bioavailability of a drug to the brain of a subject suffering from Alzheimer's disease, comprising administering to the subject a hydrophobic peptide in an amount sufficient to enhance the bioavailability of the drug to the brain of the subject.

21. The method of claim 20, wherein the hydrophobic peptide is a β-amyloid peptide derivative.

22. The method of claim 21, wherein the β-amyloid peptide derivative is selected from the group consisting of PPI-558, PPI-657, PPI-1019, PPI-578, and PPI-655.

23. The method of claim 22, wherein the β-amyloid peptide derivative is PPI-1019.

24. A method for enhancing the bioavailability of a β-amyloid peptide derivative to the brain of a subject, comprising administering to the subject the β-amyloid peptide derivative and a P-glycoprotein inhibitor, thereby enhancing the bioavailability of the β-amyloid peptide derivative to the brain of the subject.

25. The method of claim 24, wherein the β-amyloid peptide derivative is selected from the group consisting of PPI-558, PPI-657, PPI-1019, PPI-578, or PPI-655.

26. The method of claim 25, wherein the β-amyloid peptide derivative is PPI-1019.

27. The method of claim 24, wherein the P-glycoprotein inhibitor is valspodar.

28. The method of claim 24, wherein the P-glycoprotein inhibitor is cyclosporin A.

29. The method of claim 24, wherein the P-glycoprotein inhibitor is selected from the group consisting of antiarrhythmics, antibiotics, antifungals, calcium channel blockers, cancer chemotherapeutics, hormones, antiparasites, local anesthetics, phenothiazines, and tricyclic antidepressants.

30. The method of claim 24, further comprising administering to the subject a cytochrome P450 inhibitor.

31. The method of claim 24, wherein the β-amyloid peptide derivative and the P-glycoprotein inhibitor are administered simultaneously.

32. The method of claim 24, wherein the β-amyloid peptide derivative and the P-glycoprotein inhibitor are administered at different times.

33. A method for enhancing the bioavailability of a β-amyloid peptide derivative to the brain of a subject, comprising administering to the subject the β-amyloid peptide derivative and a cytochrome P450 inhibitor, thereby enhancing the bioavailability of the β-amyloid peptide derivative to the brain of the subject.

34. The method of claim 33, wherein the β-amyloid peptide derivative is selected from the group consisting of PPI-558, PPI-657, PPI-1019, PPI-578, or PPI-655.

35. The method of claim 34, wherein the β-amyloid peptide derivative is PPI-1019.

36. The method of claim 33, further comprising administering to the subject a P-glycoprotein inhibitor.

37. The method of claim 36, wherein the P-glycoprotein inhibitor is valspodar.

38. The method of claim 36, wherein the P-glycoprotein inhibitor is cyclosporin A.

39. The method of claim 36, wherein the P-glycoprotein inhibitor is selected from the group consisting of antiarrhythmics, antibiotics, antifungals, calcium channel blockers, cancer chemotherapeutics, hormones, antiparasites, local anesthetics, phenothiazines, and tricyclic antidepressants.

40. The method of claim 33, wherein the β-amyloid peptide derivative and the cytochrome P450 inhibitor are administered simultaneously.

41. The method of claim 33, wherein the β-amyloid peptide derivative and the cytochrome P450 inhibitor are administered at different times.

42. A pharmaceutical composition comprising a β-amyloid peptide derivative and a drug.

43. The pharmaceutical composition of claim 42, further comprising a P-glycoprotein inhibitor.

44. The pharmaceutical composition of claim 42, further comprising a cytochrome P450 inhibitor.

45. The pharmaceutical composition of claim 42, further comprising a pharmaceutically acceptable carrier.

46. The pharmaceutical composition of claim 42, wherein the pharmaceutically acceptable carrier is a lipid-based carrier.

47. A pharmaceutical composition comprising a β-amyloid peptide derivative and a P-glycoprotein inhibitor.

48. A pharmaceutical composition comprising a β-amyloid peptide derivative and a cytochrome P450 inhibitor.

49. A kit comprising a β-amyloid peptide derivative and instructions for administration to a subject to enhance the bioavailability of a drug in the subject.

50. The kit of claim 49, further comprising a drug.

51. The kit of claim 49, further comprising a P-glycoprotein inhibitor.

52. The kit of claim 49, further comprising a cytochrome P450 inhibitor.

53. A method for treating or preventing hepatic injury in a subject in need thereof, comprising administering to the subject a P-glycoprotein inhibitor in an amount effective to treat or prevent hepatic injury in the subject, thereby treating or preventing hepatic injury in a subject in need thereof.

54. The method of claim 53, wherein the P-glycoprotein inhibitor is selected from the group consisting of antiarrhythmics, antibiotics, antifungals, calcium channel blockers, cancer chemotherapeutics, hormones, antiparasites, local anesthetics, phenothiazines, and tricyclic antidepressants.

55. The method of claim 53, further comprising administering to the subject a cytochrome P450 inhibitor.

56. The method of claim 53, wherein the hepatic injury is selected from the group consisiting of hepatic fibrosis, hepatic cirrhosis, hepatic injury due to prolonged ethanol uptake, hepatic injury caused by a drug, hepatic injury due to carbon tetrachloride exposure.

57. A method for treating or preventing hepatic injury in a subject in need thereof, comprising:
selecting a subject in need of treatment for or prevention of hepatic injury; and
administering to the subject a P-glycoprotein inhibitor in an amount effective to treat or prevent hepatic injury in the subject, thereby treating or preventing hepatic injury in a subject in need thereof.

58. A method for modulating the levels of a hepatic enzyme in a subject, comprising:
selecting a subject in need of modulation of levels of hepatic enzymes; and
administering to the subject a P-glycoprotein inhibitor in an amount effective to modulate the levels of a hepatic enzyme in the subject.

59. A method for modulating the levels of a hepatic enzyme in a subject, comprising administering to the subject a P-glycoprotein inhibitor in an amount effective to modulate the levels of a hepatic enzyme in the subject.

60. The method of claim 59, wherein the levels of the hepatic enzyme in the subject are decreased.

61. The method of claim 59, wherein the hepatic enzyme is alanine aminotransferase.

62. The method of claim 59, wherein the hepatic enzyme is aspartate aminotransferase.

63. The method of claim 59, wherein the hepatic enzyme is γ-glutammyl transferase.

64. A pharmaceutical composition comprising a P-glycoprotein inhibitor and a drug, wherein the drug is present in an amount effective to treat a targeted condition in a subject and the P-glycoprotein inhibitor is present in an amount effective to prevent hepatic injury in the subject.

65. A kit comprising a P-glycoprotein inhibitor, a drug, and instructions for administration to a subject in an amount effective to treat a targeted condition in the subject and prevent hepatic injury in the subject.


SEQUENCE LISTING (23 sequences)

SEQ ID NO: 1 - protein, 4 aa, Artificial Sequence
  Each protein is a D-amino acid
LVFF

SEQ ID NO: 2 - protein, 5 aa, Artificial Sequence
  Each protein is a D-amino acid
LVFFA

SEQ ID NO: 3 - protein, 4 aa, Artificial Sequence
  Each protein is a D-amino acid
LVFX

SEQ ID NO: 4 - protein, 4 aa, Artificial Sequence
  Each protein is a D-amino acid
LVYF

SEQ ID NO: 5 - protein, 4 aa, Artificial Sequence
  Each protein is a D-amino acid
LVYF

SEQ ID NO: 6 - protein, 4 aa, Artificial Sequence
  Each protein is a D-amino acid
LVFY

SEQ ID NO: 7 - protein, 4 aa, Artificial Sequence
  Each protein is a D-amino acid
LVFY

SEQ ID NO: 8 - protein, 4 aa, Artificial Sequence
  Each protein is a D-amino acid
LVFA

SEQ ID NO: 9 - protein, 5 aa, Artificial Sequence
  Each protein is a D-amino acid
AVFFL

SEQ ID NO: 10 - protein, 5 aa, Artificial Sequence
  Each protein is a D-amino acid
LVYFA

SEQ ID NO: 11 - protein, 5 aa, Artificial Sequence
  Each protein is a D-amino acid
LVYFA

SEQ ID NO: 12 - protein, 5 aa, Artificial Sequence
  Each protein is a D-amino acid
LVFYA

SEQ ID NO: 13 - protein, 5 aa, Artificial Sequence
  Each protein is a D-amino acid
LVFYA

SEQ ID NO: 14 - protein, 4 aa, Artificial Sequence
  Each protein is a D-amino acid
FFVL

SEQ ID NO: 15 - protein, 4 aa, Artificial Sequence
  Each protein is a D-amino acid
AFFV

SEQ ID NO: 16 - protein, 5 aa, Artificial Sequence
  Each protein is a D-amino acid
AFFVL

SEQ ID NO: 17 - protein, 5 aa, Artificial Sequence
  Each protein is a D-amino acid
AFFLL

SEQ ID NO: 18 - protein, 5 aa, Artificial Sequence
  Each protein is a D-amino acid
LFFVL

SEQ ID NO: 19 - protein, 5 aa, Artificial Sequence
  Each protein is a D-amino acid
FFFVL

SEQ ID NO: 20 - protein, 5 aa, Artificial Sequence
  Each protein is a D-amino acid
FFFLV

SEQ ID NO: 21 - protein, 5 aa, Artificial Sequence
  Each protein is a D-amino acid
FFFFL

SEQ ID NO: 22 - protein, 5 aa, Artificial Sequence
  Each protein is a D-amino acid
AFFFL

SEQ ID NO: 23 - protein, 5 aa, Artificial Sequence
  Description of Artificial Sequence Synthetic Peptides
DDIIL